(19) United States
(12) Patent Application Publication
(10) Pub. No.: US 2009/0317797 A1
(43) Pub. Date: Dec. 24, 2009

(54) Non-Invasive, Prenatal, In-Vitro Method for Detecting the Normal Healthy Condition, the Condition of a Healthy Carrier or the Condition of a Carrier Inflicted with Cystic Fibrosis

(75) Inventors: Patrizia Paterlini, Paris (FR); Jean-Paul Bonnefont, Colombes (FR); Isabelle Desitter, Paris (FR); Ali Saker, Chevilly Larue (FR)

Correspondence Address: NIXON & VANDERHYE, PC, 901 NORTH GLEBE ROAD, 11TH FLOOR, ARLINGTON, VA, 22203, US

(73) Assignees: INSTITUT NATIONAL DE LA SANTE ET DE LA RECHERCHE MEDICALE (INSERM), PARIS (FR); ASSISTANCE PUBLIC-HOPITAUX DE PARIS, Paris (FR); UNIVERSITE RENE DESCARTES PARIS V, Paris (FR)

(21) Appl. No.: 11/795,244

(22) PCT Filed: Jan. 18, 2006
(86) PCT No.: PCT/FR06/00118

§ 371 Date: Sep. 5, 2008

(30) Foreign Application Priority Data
Jan. 18, 2005 (FR) 0500512

(51) Int. Cl. C12Q 1/68; C07H 21/04

(52) U.S. Cl. 435/6; 536/24.33

(57) ABSTRACT

The invention relates to a non-invasive, prenatal, in-vitro method for detecting the normal healthy condition, the condition of a healthy carrier or the condition of a carrier inflicted with cystic fibrosis, from the fetal cell(s) from a maternal sample, comprising the DNA of an individual to be tested. The invention also relates to oligonucleotide primers and to their use within the scope of a non-invasive, prenatal, in-vitro method for detecting the condition of a healthy carrier or of a carrier inflicted with cystic fibrosis.

The present invention relates to a non-invasive, prenatal method for detecting the normal healthy condition, the healthy carrier condition or the condition of a carrier afflicted with cystic fibrosis from fetal cell(s) derived from a maternal sample, comprising the DNA of an individual to be tested. The method of the invention may be used in an in vitro protocol for diagnosing cystic fibrosis.

The invention also relates to oligonucleotide primers and to their use in the context of a non-invasive prenatal method for detecting the normal healthy state, the healthy carrier condition or the condition of a carrier afflicted with cystic fibrosis. The invention also relates to primers and their use in the context of a method for identification, in a preparation of DNA derived from the genome or at least one cell collected from a maternal sample, of one or more markers of genetic polymorphism, demonstrating the bi-parental contribution to the DNA and as a result the fetal origin of said at least one cell.

The invention also relates to a polynucleotide and a combination of polynucleotides which may be used as primers to amplify a quantity of DNA from a biological sample, and to a kit for the non-invasive prenatal detection of cystic fibrosis.

Cystic fibrosis is an autosomal recessive disease which endangers the lives of sufferers and is most widespread among white children, affecting one newborn in 3500 in the United States of America (Kosorok, Disease, 1996), with a carrier frequency of 1 in 20 to 1 in 40 or more (Bobadilla, 2002). The disease is characterized by an imperfection in the chlorine channel which is due to alterations, more precisely to mutations in the cystic fibrosis transmembrane conductance regulatory gene (CTFR) (Cystic Fibrosis Foundation, 2003). About 1000 mutations have been discovered, in particular that corresponding to the ΔF508 allele which represents 68% of mutated alleles worldwide (Karem, 1989).

The imperfection in the chlorine channel entrains anomalies in electrolytes and macromolecular secretions from exocrine glands, inducing a risk of obstruction of the pancreatic channel in utero, pancreatic insufficiency, chronic obstructive bronchopneumopathy and recurrent respiratory infections. The average life expectancy of patients is about 30 years, but survival among patients with cystic fibrosis has increased in the last 4 decades.

More than 10 million Americans carry the ΔF508 mutation of the CFTR gene associated with cystic fibrosis and about 80% of babies born with cystic fibrosis were conceived by parents who do not have familial antecedents of the disease (Fink, Collins, 1997). Since the disease is relatively common, its first signs are early. The costs of treating this disease are high and it is inevitably fatal. Cystic fibrosis is one of the most promising diseases for genetic screening (Balinsky, 2004, Garber, Fenerty, 1991).

Reliable genetic diagnosis is carried out on fetal cells obtained by amniocentesis, chorionic villus sampling (CVS) or taking fetal blood, which suffer from a non negligible risk of miscarriage of 0.5%, 1% and 3% respectively (Ciarleglio, 2003). Thus, it is important to investigate alternative diagnostic methods, preferably non-invasive methods.

International patent application WO-A-02/088736 describes a non-invasive prenatal diagnostic method carried out from a sample of maternal blood which can in particular detect the sex of the fetus early on. The disclosure in that patent application is hereby incorporated into the present application by reference.

Previous efforts to enrich circulating fetal cells and use them for the prenatal diagnosis of cystic fibrosis have been made (Martel-Petit, 2001) and fetal DNA from maternal plasma has been used to detect a hereditary paternal mutation (Gonzalez-Gonzalez, 2002).

However, none of the methods for diagnosing cystic fibrosis has proved to be reliable and sufficiently efficient for routine application replacing invasive protocols.

The inventors have turned their attention to a non-invasive prenatal method for detecting cystic fibrosis which is reliable and sufficiently efficient to be of routine application, which does not involve taking fetal cells or tissues by biopsy and/or transgressing the placental barrier.

The invention defines means suitable for the detection of known genetic anomalies, in particular known mutations of the CFTR gene, or means adapted to the detection of unknown or undetermined mutations of the CFTR gene.

In the context of the invention, fetal cells are removed from a sample which is removed from a pregnant woman.

Thus, the invention provides a non-invasive prenatal in vitro method for detecting the normal healthy condition, the healthy carrier condition or the condition of a carrier afflicted with cystic fibrosis, from a sample of fetal cell(s) isolated from a maternal sample which has been taken, comprising DNA to be tested from an individual, said method comprising the following steps:
a) enriching a pure or diluted maternal sample, which may include cells of fetal origin, in fetal cells;
b) analyzing the retained cells and selecting cells presumed to be of fetal origin;
c) demonstrating, by genetic analysis, the fetal origin of one or more cells selected in step b); and
d) on fetal DNA of cell(s) selected in step c), investigating alleles of the CFTR gene carrying the ΔF508 mutation or other known mutations, or investigating alleles of a locus carrying a genotypical polymorphism genetically linked to an unidentified morbid mutation of the CFTR gene by means of the following steps:
   amplification of fetal DNA using pairs of primers selected for their capacity to amplify the locus which is capable of carrying the investigated known mutation on the CFTR gene or a locus comprising a genotypical polymorphism genetically linked (linkage) to the mutation of the CFTR gene during segregation;
   identifying, on the alleles corresponding to the amplified DNA fragments, the presence or absence of the investigated known mutation of the CFTR gene or of the polymorphic locus genetically linked to the CFTR gene; and
   comparing fetal alleles with alleles corresponding to control samples and determining, from observing the amplified alleles, a detection of the normal healthy condition, the healthy carrier condition or the condition of a carrier afflicted with cystic fibrosis in a test individual.

In the context of the present invention, an “individual” is the individual to be born represented by its fetal cells, for the purposes of the detection method of the invention. The detection method of the invention performs sufficiently in terms of specificity and sensitivity to be considered as a prenatal diagnostic method which is an alternative to invasive methods or at least to be included in diagnostic protocols.

Thus, starting from DNA from one or a small number of fetal cells, the method of the invention can identify the presence or absence, heterozygous or homozygous, of alleles carrying anomalies of the CFTR gene responsible for cystic fibrosis in a born individual.

The presence of homozygous alleles mutated at the CFTR gene indicates an individual who will be normal, a healthy carrier or a carrier afflicted with cystic fibrosis.

Four possibilities exist:
healthy homozygote: 2 normal alleles;
single heterozygote: 1 normal allele/1 mutated allele (ΔF508 or unknown mutation);
afflicted homozygote: 2 mutated alleles (ΔF508); composite heterozygote:
   either 1 mutated ΔF508 allele/1 mutated allele, mutation unknown;
   or 1 mutated allele, mutation unknown/1 unknown mutated allele.

There is no disease in the “healthy homozygote” and “single heterozygote” cases.

Disease is present in the “afflicted homozygote” and “composite heterozygote” cases.

The expression “healthy carrier condition” means that the individual is singly heterozygous for the mutated allele of the CFTR gene.

The expression “afflicted carrier condition” means that the individual is afflicted homozygous or composed heterozygous for the mutated allele of the CFTR gene.

The expression “normal healthy condition” means that the individual has two normal alleles.

Said detection is carried out by a direct search for a known mutation, or when the mutation of the CFTR gene responsible for cystic fibrosis is not known or has not been identified in the parents of the test individual, by an indirect method, consisting of detecting one or more polymorphism markers having a genetic linkage with the CFTR gene, said marker possibly being inherited by co-segregation with said CFTR gene through the generations.

In the context of this non-invasive prenatal in vitro method for detecting cystic fibrosis, an alteration, in particular a known mutation of the CFTR gene, is, for example, that which affects position 508 of the corresponding amino acids, resulting in deletion of three nucleotides identified as ΔF508.

When the method of the invention is implemented to investigate an unknown mutation or a mutation which has not been identified in the parents of the test individual, genetic polymorphisms must be investigated, in particular genotypical polymorphisms located upstream or downstream of the CFTR gene, on chromosome 7, which are characterized by their co-segregation with the CFTR gene over generations. An allele of such a marker is in principle always associated with the same allele of the CFTR gene.

The term “control sample” means, with respect to the biological sample providing access to fetal cells, one or more biological samples which derive respectively from the father and mother when the desired mutation of the CFTR gene is a known mutation, for example the mutation of the ΔF508 locus. When the mutation is not known, said control samples are one or more biological samples deriving respectively from the father and mother and added thereto must be a biological sample from a child of the same parentage who is afflicted with cystic fibrosis (index case). Said samples are processed to provide access to genomic DNA of the father, the mother and the child of the same parentage respectively.

The expression “maternal sample” means any biological sample taken from the mother of the test individual by a non-invasive method, said sample comprising fetal cells as well as other types of cells.

In a specific example, the maternal sample may be maternal blood taken from a pregnant woman, including fetal cells as well as other circulating cells such as leukocytes. Step a) of the method of the invention will in this case consist of separating the fetal cells from other circulating cells.

In another implementation of the invention, the maternal sample is collected from the pregnant woman from the neck of the uterus either by washing with a solution (for example physiological) or by collecting mucus without washing, without transgressing the amniotic sac. In principal, the collected sample contains fetal cells.

The method for detecting cystic fibrosis of the invention can be understood to be a non-invasive in vitro method which allows the pre-disposition of an individual to cystic fibrosis to be assessed, by identifying in that individual the presence or absence, heterozygous or homozygous, of alleles carrying anomalies of the CFTR gene responsible for cystic fibrosis.

Apart from having available the required information regarding the DNA of control samples before undertaking the detection of the invention on the collected fetal DNA, the control samples are processed in steps c) and d) of the method of the invention in the same way as a fetal DNA sample.

In a particular implementation of the invention, the quantity of DNA which is amplified is a small quantity of DNA, advantageously less than 5 pg, for example of the order of 2 pg. The amplification primers must then be defined to allow amplification of a small quantity of DNA, for example a quantity of DNA corresponding to that of a single cell or a reduced number of fetal cells.

According to a specific example of the detection method, the amplification step of step c) and/or the amplification step of step d) comprises at least one amplification phase, for example a first amplification phase carried out with external primers and a second amplification phase carried out with internal primers.

In a specific example of the invention, sample DNA from the test individual derives from the genome of at least one fetal cell.

In a specific example, DNA from the cell sample derives from the genome of at least one isolated fetal cell, in particular from the genome of a small number of cells, from 1 to 20 cells, more particularly from 1 to 10 cells, for example the genomes from 1, 2 or 3 cells, preferably at least three cells (see Example 4 below) and advantageously from the genome of individually isolated cells.

In a specific example, the sample DNA derives from the genome of a single fetal cell or from a number of fetal cells equal to or less than 20, more particularly less than 10. Preferably, the number of fetal cells is at least three.

If detection on the DNA from a single cell does not provide a pertinent result, in particular if amplification is carried out by PCR and one of the two alleles of the gene carrying the desired marker is not amplified or is not detected (a situation known as allele drop-out) because of the quantity of DNA employed, the number of fetal cells from which the DNA to be tested is to be recovered can be increased and then used in the form of a pool. In particular, 2, 3 or, for example, between 2 and 20 cells would be used, preferably at least three cells, advantageously 3 to 20 cells (limits included), see Example 4 below (absence of ADO).

After having determined the alleles of the CFTR gene of the fetal DNA or the alleles of the locus carrying the polymorphism markers having a linkage with the CFTR gene, a comparison is carried out with alleles from the father, the mother and if necessary the child from the same parentage afflicted with cystic fibrosis (denoted the index case) to find out if, in the DNA sample from the test individual being studied, there is the homozygous presence of an allele characteristic of a genetic alteration linked to cystic fibrosis, the heterozygous presence of an allele characteristic of a genetic alteration linked to cystic fibrosis or the homozygous absence of an allele characteristic of a genetic alteration linked to cystic fibrosis.

To be able to detect cystic fibrosis in an individual in vitro, the fetal DNA from which is tested, in a first detection phase, in step c) of the method of the invention, it is necessary to demonstrate the fetal origin of the DNA. This determination of the fetal nature of the DNA is henceforth termed genotyping in the present application. This step is carried out using genetic polymorphism markers. It is necessary for the genetic polymorphism markers used to be informative in that they can differentiate the allele of each marker provided by the father and that provided by the mother. Thus, the maternal and paternal polymorphism markers must be distinct from each other.

In a particular implementation, the invention concerns a method for detecting the healthy carrier condition or a carrier afflicted with cystic fibrosis, characterized in that the informative primers used in step c) are derived from the sequence of a chromosome selected from chromosome 16, chromosome 21 and chromosome 7.

In a particular implementation of the invention, the primers used in step c) are derived from the sequence of chromosome 7 and are close to the morbid allele for cystic fibrosis. The morbid allele designated here is the allele of the CFTR gene (located on chromosome 7) which carries the anomaly responsible for cystic fibrosis.

Prior to the genotyping step c) and to the step for investigating alleles of the CFTR gene or alleles of a locus carrying a genetic polymorphism genetically linked to an abnormal allele of the CFTR gene, the invention requires the following steps to be carried out:
a) enriching a pure or diluted maternal sample, which may include cells of fetal origin, in fetal cells;
b) analyzing cells retained during step a) to obtain a presumption of their fetal or maternal origin.

In the context of the non-invasive prenatal method for detecting cystic fibrosis of the invention, any type of technique may be used firstly for enrichment of a pure or diluted maternal sample in fetal cells, which may include cells of fetal origin (step a)), and secondly to analyze cells retained during step a) to obtain a presumption of their fetal or maternal origin (step b)).

Various methods which can be used for step a) and b) will be envisaged and illustrated below, after the description of steps c) and d). In a specific example of the invention, cellular genomes are individually analyzed during step c). During step d), an analysis of the cellular genomes is either carried out on the genome of each cell taken individually or on the pooled genomes from several fetal cells.

More particularly, genotyping step c) comprises or consists of identification, on a preparation of DNA derived from the genome of a single collected cell (individual cell), of one or more genetic polymorphism markers or a combination of said markers, demonstrating the bi-parental contribution to the DNA and as a result, the fetal origin of said at least one cell.

In the context of the present non-invasive prenatal method for detecting cystic fibrosis, the term “genetic polymorphism marker” should be understood to include any characteristic identifiable on the DNA the presence of which is correlated to a particular genotype and if appropriate to a particular phenotype. The markers used during step c) can distinguish paternal DNA from maternal DNA and thus demonstrate the bi-parental composition of the fetal DNA. Thus, it may be sufficient that they are correlated to a particular genotype.

Examples which may be cited are restriction fragment length polymorphism markers (RFLP), SNP markers (single nucleotide polymorphism), micro-satellite markers, VNTR (variable number of tandem repeats) markers or STR (short tandem repeats).

Micro-satellite markers are particularly preferred for the characterization of cells and carrying out the prenatal diagnosis. In one implementation of the invention, at least one polymorphism marker to be identified is a micro-satellite marker, a VNTR (variable number of tandem repeats) marker, a SNP (single nucleotide polymorphism) marker or an STR (short tandem repeat) marker. These have the advantage of being identifiable by amplification using specific primers. Micro-satellite, VNTR or STR markers are composed of repeat sequences in tandem, usually polyCA/GT motifs. Allelic variations due to the variation in the number of repetitions are readily detected by PCR type amplification using primers corresponding to the unique sequences flanking the micro-satellite. A physical map of these micro-satellite markers and the sequence of their associated primers are described by Dib et al (Dib, C, Faure, S, Fizames, C, Samson, D, Drouot, N, Vignal, A, Missasseau, P, Marc, S, Hazan, J, Seboun, E, Lathrop, M, Gyapay, G, Morissette, J, and Weissenbach; J A comprehensive genetic map of the human genome based on 5264 micro-satellites Nature 1996 380: 152-154). Using this methodology, demonstration of a bi-parental contribution to the genotype of the analyzed cells can definitely establish the fetal origin of the analyzed cells.

In order to demonstrate the fetal origin or, in contrast, the maternal origin of a single collected cell, in a particular implementation, it may be sufficient to search for a marker or a combination of markers or an allelic assay of these markers which are distinguished from those of the genome of maternal cells. In particular, the investigation on the genome of said collected cell may be carried by a marker or a combination of markers specific for the DNA of paternal cells. Their presence is necessarily the signature of a fetal origin of the cell in question.

In a particular implementation of the invention, step c) may comprise:
i) amplifying individually taken DNA from one or more selected cells using primers termed informative primers, capable of amplifying predetermined genetic polymorphisms to distinguish maternal alleles from paternal alleles in order to recognize the fetal genome by the presence of a paternal allele and a maternal allele in each selected cell;
ii) comparing alleles of DNA from said cells with the corresponding parental alleles;
iii) selecting DNA from cell(s) comprising a maternal allele and a paternal allele for the identified genetic polymorphisms, demonstrating the fetal origin of the DNA from the cell(s).

An example of the conditions for carrying out the amplifications are as follows: 5 min 94° C., 40 x (30 s, 94° C.; 30 to 45 s, 55° C. to 61° C.; 30 s 72° C.), 5 min 72° C.

In a specific example, two amplification phases may be carried out as follows: a first phase for amplification of the preparation of DNA derived from the genome of a single collected cell is carried out with one or more nucleotide primers termed external primers with respect to the target sequence and a second amplification phase is carried out on the amplification product obtained by the preceding step with one or more nucleotide primers termed internal or nested primers with respect to said external primers. The above external and internal primers are termed informative, i.e. capable of amplifying predetermined genetic polymorphisms as being distinct and characteristic on maternal alleles and paternal alleles, to allow identification of typical investigated genetic polymorphisms of fetal alleles.

The primers are synthetic oligonucleotides the sequence of which is determined to allow amplification of a DNA sequence comprising informative markers for the fetal nature of the test cell.

As a consequence, the primers used to carry out step c) are termed informative of the fetal nature of the cell or, in a particular implementation of the invention, said primers are termed informative of the bi-parental contribution of the test cell.

Informative primers advantageously result in amplification of DNA fragments comprising several polymorphisms, preferably a large number of polymorphisms. The polymorphisms are identified on DNA sequences of interest, in particular on chromosome 7, in available databases (NCBI for example). From these data, oligonucleotides are identified which allow amplification under the conditions of the invention, to allow efficient, sensitive and specific genotyping or detection.

Said informative primers may be specifically identified for a family (father and mother of the test individual) by comparative genetic analysis of the genomes of the father and mother.

Alternatively, said informative primers may be primers for which it has been shown that they are capable of amplifying DNA sequences comprising genetic polymorphism markers shared by several families, or consensual, regarding the informative nature of the paternal or maternal origin of an allele of a gene.

This is the case with the primers identified below, which can reveal the bi-parental contribution to the DNA of the test cell to demonstrate its fetal origin.

The external primers, termed informative primers, which can be used in step c) are selected from the following pairs of primers, where F signifies “forward” and hybridizes with the 3′-5′ oriented DNA strand in the target sequence and R signifies “reverse” and hybridizes with the 5′-3′ oriented DNA strand in the target sequence:

(SEQ ID NO: 1)

F: 5′-AAAAACCCTGGCTTATGC-3′;

(SEQ ID NO: 2)

R: 5′-AGCTACCATAGGGCTGGAGG-3′;

(SEQ ID NO: 5)

F: 5′-GGAATCTGTTCTGGCAATGGAT-3′;

(SEQ ID NO: 6)

R: 5′-TTGCAATGAGCCGAGATCCTG-3′;

(SEQ ID NO: 9)

F: 5′-CAGATGCTCGTTGTGCACAA-3′;

(SEQ ID NO: 10)

R: 5′-ATACCATTTACGTTTGTGTGTG-3′;

(SEQ ID NO: 13)

F: 5′-TGACAGTGCAGCTCATGGTC-3′;

(SEQ ID NO: 14)

R: 5′-GGTCATTGGTCAAGGGCTGCT-3′;

(SEQ ID NO: 17)

F: 5′-TTGACATTCTTCTGTAAGGAAGA-3′;

(SEQ ID NO: 18)

R: 5′-AGGCTTGCCAAAGATATTAAAAG-3′;

(SEQ ID NO: 21)

F: 5′-TTGTGAATAGTGCTGCAATG-3′;

(SEQ ID NO: 22)

R: 5′-ATGTACACTGACTTGTTTGAG-3′;

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′.

The internal or nested primers, termed informative primers, used in step c) are selected from the following primer pairs:

(SEQ ID NO: 3)

F: 5′-CTTGGGGACTGAACCATCTT-3′;

(SEQ ID NO: 4)

R: 5′-AGCTACCATAGGGCTGGAGG-3′;

(SEQ ID NO: 7)

F: 5′-AAAGGCCAATGGTATATCCC-3′;

(SEQ ID NO: 8)

R: 5′-GCCCAGGTGATTGATAGTGC-3′;

(SEQ ID NO: 11)

F: 5′-GATCCCAAGCTCTTCCTCTT-3′;

(SEQ ID NO: 12)

R: 5′-ACGTTTGTGTGTGCATCTGT-3′;

(SEQ ID NO: 15)

F: 5′-GGATAAACATAGAGCGACAGTTC-3′;

(SEQ ID NO: 16)

R: 5′-AGACAGAGTCCCAGGCATT-3′;

(SEQ ID NO: 19)

F: 5′-CCCTCTCAATTGTTTGTCTACC-3′;

(SEQ ID NO: 20)

R: 5′-GCAAGAGATTTCAGTGCCAT-3′;

(SEQ ID NO: 23)

F: 5′-ATGTACATGTGTCTGGGAAGG-3′;

(SEQ ID NO: 24)

R: 5′-TTCTCTACATATTTACTGCCAACA-3′;

(SEQ ID NO: 31)

F: 5′-CCTTGGGCCAATAAGGTAAG-3′;

(SEQ ID NO: 32)

R: 5′-AGCTACTTGCAGTGTAACAGCATTT-3′;

(SEQ ID NO: 35)

F: 5′-CTGATTCATAGCAGCACTTG-3′;

(SEQ ID NO: 36)

R: 5′-AAAACATTTCCATTACCACTG-3′.

Step d) which follows step c) must allow detection of cystic fibrosis from fetal cells retained at the end of step c). Thus, known mutations of the CFTR gene or polymorphisms markers forming a linkage with the mutated allele of the CFTR gene the presence of which is witness to alterations in the DNA of the CFTR gene are investigated.

It is known that in the prior art, these mutations affect the CFTR gene, mainly in said region of the F508 locus where a mutation by deletion of three nucleotides leads to the production of a CFTR gene mutated in the “508” position. As a consequence, amplified fragments of the CFTR gene using the primers of the invention are those which include the ΔF508 mutation. The inventors have thus defined oligonucleotides, which can be used as amplification primers to carry out step d) to amplify a DNA sequence comprising the F508 locus and to detect the mutation of said locus.

Other mutations may, however, occur on the CFTR gene in regions other than the F508 locus. Thus, the inventors have defined other primer oligonucleotides capable of amplifying DNA sequences carrying a genetic polymorphism forming a linkage (or genetic connection) with a locus carrying other mutations termed “unidentified” associated with cystic fibrosis. This amplification is carried out in the context of the cystic fibrosis detection method. These primers have been identified from DNA from chromosome 7.

Step d) of the method of the invention may thus be carried out using primer pairs selected from the following:
for ΔF508 type mutations:
   the external ΔF508 out primer pair (F: 5′-TGGAGCCTTCAGAGGGTAAA-3′ (SEQ ID NO: 25); R: 5′-TGCATAATCAAAAAGTTTTCACA-3′ (SEQ ID NO: 26)), used alone or during the first amplification phase of step d) when it is carried out; and
   the ΔF508 internal in primer pair (F: 5′-TCTGTTCTCAGTTTTCTGG-3′ (SEQ ID NO: 27); R: 5-TCTTACCTCTTCTAGTTGGC-3′ (SEQ ID NO: 28)), used during the second amplification phase of step d) or alone in the case of a single amplification of step d);
for the other indeterminate mutations of the ΔF508 locus, primers identified in chromosome 7 amplifying a DNA fragment forming a linkage with the CFTR gene:

(SEQ ID NO: 1)

F: 5′-AAAAACCCTGGCTTATGC-3′;

(SEQ ID NO: 2)

R: 5′-AGCTACCATAGGGCTGGAGG-3′;

(SEQ ID NO: 5)

F: 5′-GGAATCTGTTCTGGCAATGGAT-3′;

(SEQ ID NO: 6)

R: 5′-TTGCAATGAGCCGAGATCCTG-3′;

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′;

(SEQ ID NO: 3)

F: 5′-CTTGGGGACTGAACCATCTT-3′;

(SEQ ID NO: 4)

R: 5′-AGCTACCATAGGGCTGGAGG-3′;

(SEQ ID NO: 7)

F: 5′-AAAGGCCAATGGTATATCCC-3′;

(SEQ ID NO: 8)

R: 5′-GCCCAGGTGATTGATAGTGC-3′;

(SEQ ID NO: 31)

F: 5′-CCTTGGGCCAATAAGGTAAG-3′;

(SEQ ID NO: 32)

R: 5′-AGCTACTTGCAGTGTAACAGCATTT-3′;

(SEQ ID NO: 35)

F: 5′-CTGATTCATAGCAGCACTTG-3′;

(SEQ ID NO: 36)

R: 5′-AAAACATTTCCATTACCACTG-3′.

In a particular implementation of the invention, in the context of the non-invasive prenatal method for detecting cystic fibrosis using the approach termed “indirect”, in which the investigated anomaly in the CFTR gene is unknown, steps c) and d) are carried out under the following conditions:
the amplification of steps c) and d) is combined and carried out using informative amplification primers which are capable of amplifying a locus comprising a genotypical polymorphism genetically linked (linkage) to the CFTR gene during segregation, for example primers identified in the present application on chromosome 7; and
the comparison with the control samples of step d) comprises comparing the alleles identified from fetal DNA with the corresponding paternal alleles, the corresponding maternal alleles and the alleles corresponding to a child of the same parentage afflicted with cystic fibrosis.

In accordance with this “indirect” detection mode, in the context of the invention, at least one phase for amplification of fetal DNA, maternal DNA, paternal DNA and DNA from a child of the family is carried out with one or more pairs of primers selected from:

(F: 5′-AAAAACCCTGGCTTATGC-3′ SEQ ID NO: 1;
R: 5′-AGCTACCATAGGGCTGGAGG-3′ SEQ ID NO: 2),
(F: 5′-GGAATCTGTTCTGGCAATGGAT-3′ SEQ ID NO: 5;
R: 5′-TTGCAATGAGCCGAGATCCTG-3′ SEQ ID NO: 6),
(F: 5′-AAGTAATTCTCCTGCCTCAG-3′ (SEQ ID NO: 29);
R:
5′-AGCTACTTGCAGTGTAACAGCATTT-3′ (SEQ ID NO: 30));
(F: 5′-GAATTATAACCGTAACTGATTC-3′ (SEQ ID NO: 33);
R: 5′-GAGATAATGCTTGTCTGACTTC-3′ (SEQ ID NO: 34));
(F: 5′-CTTGGGGACTGAACCATCTT-3′ SEQ ID NO: 3;
R: 5′-AGCTACCATAGGGCTGGAGG-3′ SEQ ID NO: 4),
(F: 5′-AAAGGCCAATGGTATATCCC-3′ SEQ ID NO: 7;
R: 5′-GCCCAGGTGATTGATAGTGC-3′ SEQ ID NO: 8),
(F: 5′-CCTTGGGCCAATAAGGTAAG-3′ (SEQ ID NO: 31);
R:
5′-AGCTACTTGCAGTGTAACAGCATTT-3′ (SEQ ID NO: 32));
(F: 5′-CTGATTCATAGCAGCACTTG-3′ (SEQ ID NO: 35);
R: 5′-AAAACATTTCCATTACCACTG-3′ (SEQ ID NO: 36)).

In other words, in this case, the primers used can both demonstrate the fetal origin of the test cell and detect cystic fibrosis, by identifying the presence or absence of at least one allele carrying a genetic polymorphism forming a linkage with the morbid allele for cystic fibrosis.

Primers other than the foregoing may be defined and used to identify the markers investigated in step c) of the method of the invention.

In particular, in a further implementation of the indirect detection method of the invention, to counter the risk that one of the two alleles of the gene carrying the investigated marker is not amplified or not detected (risk of allele drop-out, ADO), the primers used to carry out the amplification of step c) are selected to amplify DNA sequences beyond chromosome 7. Said primers are designed to be informative, in accordance with the above description, to allow paternal alleles to be distinguished from maternal alleles in fetal cells. Next, to carry out step d), the primers selected for amplification may be primers identified from DNA from chromosome 7, in the context of that described in the present description.

To define the informative primers for genotyping the test cell, oligonucleotides are selected which are capable of amplifying, for example by PCR, sequences comprising distinct genetic polymorphism markers in cells from the father and mother: the revealed amplification products are represented by peaks of the gene concerned in DNA from the fetal cell which are distinct for the paternal allele and for the maternal allele.

Preferably, the oligonucleotides are elongated over the sequence to be amplified so that the size of the amplification product is less than 1000 bp, or even less than 800 bp. The limitation in the size of the amplified sequence takes into account the fact that the quantity of DNA from the test cell, when that cell is unique, is low and is obtained from a fixed cell which thus may be altered. The selected primers must allow amplification of small quantities of DNA, for example quantities below 5 pg, in particular about 2 pg.

In the context of the method for detecting the healthy carrier condition or the condition of a carrier afflicted with cystic fibrosis of the invention to carry out step d), primers capable of amplifying the ΔF508 locus may also or alternatively be used to investigate the ΔF508 allele characteristic of the most frequent mutations of the CFTR gene in the case of cystic fibrosis.

Said primers which are capable of amplifying the ΔF508 locus are:
the external ΔF508 out primer pair (F: 5′-TGGAGCCTTCAGAGGGTAAA-3′ (SEQ ID NO: 25); R: 5′-TGCATAATCAAAAAGTTTTCACA-3′ (SEQ ID NO: 26), used during the first amplification phase of step d); and
the internal ΔF508 in primer pair F: 5′-TCTGTTCTCAGTTTTCTGG-3′ (SEQ ID NO: 27; R: 5-TCTTACCTCTTCTAGTTGGC-3′ (SEQ ID NO: 28) used during the second amplification phase of step d).

In general, primers which may be used to carry out the invention may be constituted by variant oligonucleotides derived from polynucleotides the sequences of which have been given above.

A variant oligonucleotide has, for example, at least 60% identity, preferably 80%, more preferably 95% identity or more with the primer from which its sequence derives.

Said variant oligonucleotide has one or more sequence modifications compared with the primer from which its sequence derives, in particular one or more deletion, addition or substitution type modifications.

In a specific example, said variant oligonucleotide is the same size as the primer from which its sequence derives.

In another specific example, the length of said variant oligonucleotide is less than that of the primer from which its sequence derives, for example 10% to 20% shorter, or optionally it may be longer, for example by 10% to 40%, in particular 30% to 40% longer than the sequence of the primer from which it derives.

Variant primers of the oligonucleotides cited above may, for example, be derived from these oligonucleotides by addition of one or more nucleotides at the 5′ end of said sequences.

Said oligonucleotide has the same properties as the primer from which it derives if it can be used as a primer to amplify DNA from a biological sample, in particular to amplify sequences comprising markers of fetal origin in a cell, or to amplify sequences comprising genetic polymorphism markers forming a linkage with the altered CFTR gene characteristic of cystic fibrosis.

If appropriate, the oligonucleotides which can be used in the context of the invention may be associated with markers, for example added at the 5′ or 3′ end of their sequence.

In the context of the method for detecting the healthy carrier condition or the condition of a carrier afflicted with cystic fibrosis of the invention, when there is no child afflicted with cystic fibrosis with the same parentage, the pairs of primers used first during step d) will be primers capable of amplifying the ΔF508 locus to investigate the ΔF508 allele or other known mutations directly detectable by the method of the invention.

In a particular mode of the invention, prior to demonstrating the fetal or maternal origin of a collected cell, said collected cell is lysed and together with its genome it is pre-amplified, for example using generic primers covering all of the possible sequences using known primer extension pre-amplification methods (PEP) (Zhang, L, Cui, X, Schmitt, K, Hubert, R W N, Arnheim, M Whole genomic amplification from a single cell: implications for genetic analysis PNAS 1992, 89: 5847-5851) or the DOP-PCR method. These methods can amplify the whole genome of a single cell. The pre-amplified DNA preparation obtained and derived from DNA from a single cell may then be purified and used as genetic material for the specific detection of genetic markers or markers for polymorphism in the context of steps c) and/or d) of the method of the invention.

In a specific example of the invention, the fetal or maternal origin of a collected cell is demonstrated by amplification of genetic or polymorphism markers or a combination of said markers, starting from the preparation of pre-amplified DNA derived from DNA from a single cell. The genetic polymorphism markers capable of demonstrating the bi-parental contribution to the fetal DNA are identified by prior analysis of paternal and maternal DNA using primers specifically identified for characterizing paternal DNA and for characterizing maternal DNA or, when consensual primers exist, to determine the paternal or maternal origin of an allele of a gene, after verifying that the primers in question are pertinent to the analysis of available controlled samples.

In the context of the present invention, any technique which allows specific amplification of a given nucleic acid may be used. Examples which may be cited are PCR (polymerase chain reaction) or isothermal amplification methods such as TMA (transcription mediated amplification), NASBA (nucleic acid sequence based amplification), 3SR (self sustained sequence replication) or strand displacement amplification.

The amplification methods, in particular PCR, are sufficiently sensitive to be carried out from at least a fifth of the pre-amplified DNA preparation. As a consequence, each pre-amplified DNA preparation from a collected cell may be used for amplification of at least five different markers.

In particular, amplifications carried out by PCR allow the detection of genetic polymorphism markers (for example 1, 2, 3, 4 or 5 markers), to demonstrate the fetal origin of the isolated cells. PCR amplification can also detect genetic markers located upstream, downstream or on the CFTR gene which is characteristic of cystic fibrosis. The sequences which are capable of carrying the mutation, in particular deletion or repetition of a DNA sequence, are amplified and the amplification products are separated according to size, for example by electrophoresis. The presence of deletions or, in contrast, repetitions, is detected by the presence of an amplification product smaller or larger in size than the amplification products which do not carry a deletion or repetition, which may be represented by a difference in the size of the peaks corresponding to said amplification products.

The amplification products may also be sequenced, in particular to accurately characterize the genetic markers, or to pinpoint point mutations.

In a further implementation of the method of the invention, steps c) and d) are carried out by hybridizing all or part of the pre-amplified DNA preparation or DNA preparation amplified with specific DNA probes. The DNA probes are selected so that they specifically hybridize with markers for their identification or on sequences carrying the markers to be investigated. Hybridization of the probes on the markers may be detected using conventional techniques for detecting hybridization complexes of nucleic acids of the slot blot, Southern blot or, advantageously, using DNA chips or microarrays or macroarrays (Sambrook et al, Molecular Cloning: A Laboratory Manual, 2001, 3^{rd} edition, Cold Spring Harbor Laboratory Press, Cold Spring Harbor, N.Y.).

Hence, in one implementation of the invention, the specific DNA probes for markers to be identified are fixed on a support forming a DNA chip or microarray or macroarray. The pre-amplified or amplified DNA preparation is, for example, labeled using a radioactive marker or fluorescent marker, and brought into contact with the DNA chip or microarray or macroarray comprising the specific probes. The hybridization intensity is measured for each spot containing a specific probe, thereby determining with great sensitivity the presence of the desired markers on the DNA of a collected cell.

When the method for detecting the normal healthy condition, the healthy carrier condition or the condition of a carrier afflicted with cystic fibrosis of the invention is carried out prenatally, a sample is removed from the pregnant woman. In a particular implementation of the invention, a maternal sample is removed early on during the pregnancy (for example at about the fifth week of pregnancy). However, removal of the maternal sample and the diagnostic method of the invention may be carried out at any time from the start to the end of the pregnancy. In a particular implementation of the invention, removal and the diagnostic method are carried out between the 7^{th} and 15^{th} week of pregnancy. In a further implementation, the removal and the diagnostic method are carried out between the 10^{th} and 15^{th} week of pregnancy.

In the context of removal of maternal blood, in general between 3 and 20 milliliters of maternal blood are taken, preferably between 5 and 10 milliliters. If for practical reasons between 3 and 20 milliliters of maternal blood are removed, it should be understood that the subsequent analysis of isolated fetal cells described in the context of the invention may be carried out on a more limited volume of blood, for example 1 to 10 milliliters, preferably 2 to 5 milliliters. To increase the sensitivity of the diagnosis, it is possible to take several independent samples to repeat the diagnosis on different independent samples. Further, in a preferred implementation of the invention, it is possible to remove in parallel a sample from the father and a sample of cells from the mother, for example by removing buccal cells by scraping, or by removing blood, optionally before pregnancy, and to carry out on the removed material an investigation of specific markers for the paternal genome and the maternal genome. This parallel study can identify specific genetic markers for the father and mother which could be used to demonstrate the fetal origin of cells isolated using the implementations described in the present application.

In the context of step a) for enriching a pure or diluted maternal sample which may include cells of fetal origin in fetal cells, any technique may be employed, in particular: filtration; gradient separation; immunological selection (positive; the immunologically marked cells are the cells of interest, i.e. fetal cells; or negative; the immunologically marked cells are not the pertinent cells); proliferation of cells of interest, i.e. fetal cells; lysis of non pertinent cells.

The epithelial fetal cells (trophoblasts) circulating in the blood have a larger diameter than the maternal leukocyte and erythrocyte cells and may be isolated using filtration processes adapted from those described to isolate pathogenic cells circulating in the blood such as those described in FR-A-2 782 730. The teaching of that patent application is hereby incorporated into the present application by reference.

In a specific example, step a) consists of filtering a pure or diluted maternal sample which may comprise cells of fetal origin to concentrate on a filter, according to size, certain cells including cells of fetal origin, and step b) consist of analyzing cells retained on the filter and selecting cells presumed to be of fetal origin.

When the cell sample derives from maternal blood, filtration can enrich and separate fetal cells from blood cells, in particular maternal leukocytes.

Prior to filtration step a), any process which can enrich the cell population derived from the sample in cells of fetal origin may be employed. In one implementation of the invention, the fetal cell population is enriched by sorting the cells as a function of the expression of surface markers expressed by the fetal cells to reduce the proportion of cells of maternal origin. Examples of cell sorting techniques are FACS, magnetic affinity column sorting (MACS) or any technique which can enrich one cell type on the basis of physical characteristics (density) or structural characteristics (in particular specific antigens).

To facilitate filtration, in one particular implementation, before the filtration step, the sample may be diluted in a filtration solution, said filtration solution consisting of a reagent for fixing nucleated cells and/or for lysing red blood cells. One example of a filtration solution comprises a detergent capable of degrading the membrane of red blood cells, such as saponin, and a fixative capable of stabilizing the membrane of nucleated cells such as formaldehyde.

In one preferred implementation, the maternal sample is diluted by about 10 to 100 times in the filtration solution.

The pure or diluted sample is filtered using a porous filter which can separate the cells by size. In the context of a sample from maternal blood, the porosity of the filter is selected so as to allow blood elements to pass through, in particular erythrocytes, platelets and maternal leukocytes, and to retain certain nucleated cells, in particular wide cells (epithelial or hematopoietic precursors) of maternal and fetal origin.

A filter with a porosity in the range 6 to 15 μm and a density suitable for the selected porosity which can retain cells while avoiding blocking them during filtration may in particular be used. In accordance with a specific example, the filter has substantially cylindrical pores with a diameter of about 8 μm and a density in the range 5×10⁴ to 5×10⁵ pores/cm². In a specific example, the filter used is calibrated so that all of the pores have substantially the same diameter. One example of a filter which can be used in the process of the invention is a calibrated polycarbonate type filtration membrane of the “track-etched membrane” type with a pore density of 1×10⁵ pores/cm², a thickness of 12 μm and a pore size of 8 μm, such as that sold by Whatman®.

Step a) of the method of the invention is based on the existence and development of a specific device known as the ISET (isolation by size of epithelial tumor cells) described in EP-A-0 513 139 and comprising, on a frame:
a porous filter which can retain certain cells (circulating as taken from maternal blood) according to size, mounted between two clamping devices, respectively upstream and downstream in the direction of filtration, and acting as seals;
the upstream block comprising storage and/or pre-treatment means for the samples for analysis;
the downstream block comprising perforations facing the storage means to collect waste;
forced filtration means.

Thus, the invention also pertains to an adaptation and use of a device of this type to the filtration of fetal cells present in a maternal sample removed for the purposes of the non-invasive prenatal method for detecting cystic fibrosis.

The term “adaptation and use” means:
incorporating into the device a filter with a porosity, preferably calibrated, which can retain cells with a mean diameter of more than 8 μm, preferably more than 10 μm, more preferably more than 15 μm. A filter with a mean porosity of 8 μm has been shown have the desired characteristics;
adaptation of the density of the pores to the filter, as a function of the size of the pores to optimize the filtration capacity;
adaptation of the pressure applied to the filtration means to conservation of the physical integrity of the fetal cells under investigation;
adaptation of the maternal sample dilution medium to conserving the integrity and viability of the cells under investigation.

Step a) of the method of the invention may include using an ISET type filtration device to isolate fetal cells from a maternal cell sample, comprising a filter with a mean porosity in the range 6 μm to 15 μm, preferably about 8 μm.

Step a) may also comprise using an ISET type device in which the filter has pores with a diameter of about 8 μm and a pore density in the range 5×10⁴ to 5×10⁵.

Step a) may finally include using an ISET type filtration device in which the filtration under-pressure applied is in the range 0.05 bars to 0.8 bars, preferably about 0.1 bars.

In a specific example of the invention, the cells retained on the filter during step a) are collected individually.

The term “individual collection of cells” means any method which can collect a specific individual cell retained during step a) for its subsequent analysis independently of other cells retained during step a).

More particularly, cells retained during step a) may be collected individually, in particular by microdissection.

In one specific example, step a) consists of filtration, and microdissection consists, for example, of laser cutting the portion of a filtration membrane on which a cell is retained or of detaching the cell using a laser then recovering the single collected cell in a suitable tube. This is then capable of undergoing the various analyses of steps b), c) and d) described in the invention.

In a specific example, cells retained during step a) may be analyzed in situ during step b) without individual collection of the cells. This analysis is carried out by a cytological, immunological or molecular method, for example one of the following: FISH, in situ PCR, PNA, PRINS.

The cells retained in step a) are collected then analyzed in situ during step b).

Collecting cells individually can advantageously target the genetic analysis on the genome of a single cell. It can also detect cystic fibrosis on the genome of a single cell the fetal origin of which has been demonstrated by genetic analysis. Thus, using this implementation of the invention, pure genetic material is obtained, i.e. derived from a single cell, which can be used for steps c) and d) of the method of the invention.

In the context of the present non-invasive prenatal method for detecting cystic fibrosis, the use of various suitable primers for carrying out step c) or step d) or both steps has been illustrated. In a variation of this method, other primers may be used which, for example, consist of variant oligonucleotides the sequence of which is derived from that of a primer selected from:

(SEQ ID NO: 1)

F: 5′-AAAAACCCTGGCTTATGC-3′;

(SEQ ID NO: 2)

R: 5′-AGCTACCATAGGGCTGGAGG-3′; or

(SEQ ID NO: 5)

F: 5′-GGAATCTGTTCTGGCAATGGAT-3′;

(SEQ ID NO: 6)

R: 5′-TTGCAATGAGCCGAGATCCTG-3′; or

(SEQ ID NO: 9)

F: 5′-CAGATGCTCGTTGTGCACAA-3′;

(SEQ ID NO: 10)

R: 5′-ATACCATTTACGTTTGTGTGTG-3′; or

(SEQ ID NO: 13)

F: 5′-TGACAGTGCAGCTCATGGTC-3′;

(SEQ ID NO: 14)

R: 5′-GGTCATTGGTCAAGGGCTGCT-3′; or

(SEQ ID NO: 17)

F: 5′-TTGACATTCTTCTGTAAGGAAGA-3′;

(SEQ ID NO: 18)

R: 5′-AGGCTTGCCAAAGATATTAAAAG-3′; or

(SEQ ID NO: 21)

F: 5′-TTGTGAATAGTGCTGCAATG-3′;

(SEQ ID NO: 22)

R: 5′-ATGTACACTGACTTGTTTGAG-3′; or

(SEQ ID NO: 25)

F: 5′-TGGAGCCTTCAGAGGGTAAA-3′;

(SEQ ID NO: 26)

R: 5′-TGCATAATCAAAAAGTTTTCACA-3′; or

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′;

(SEQ ID NO: 3)

F: 5′-CTTGGGGACTGAACCATCTT-3′;

(SEQ ID NO: 4)

R: 5′-AGCTACCATAGGGCTGGAGG-3′; or

(SEQ ID NO: 7)

F: 5′-AAAGGCCAATGGTATATCCC-3′;

(SEQ ID NO: 8)

R: 5′-GCCCAGGTGATTGATAGTGC-3′; or

(SEQ ID NO: 11)

F: 5′-GATCCCAAGCTCTTCCTCTT-3′;

(SEQ ID NO: 12)

R: 5′-ACGTTTGTGTGTGCATCTGT-3′; or

(SEQ ID NO: 15)

F: 5′-GGATAAACATAGAGCGACAGTTC-3′;

(SEQ ID NO: 16)

R: 5′-AGACAGAGTCCCAGGCATT-3′; or

(SEQ ID NO: 19)

F: 5′-CCCTCTCAATTGTTTGTCTACC-3′;

(SEQ ID NO: 20)

R: 5′-GCAAGAGATTTCAGTGCCAT-3′: or

(SEQ ID NO: 23)

F: 5′-ATGTACATGTGTCTGGGAAGG-3′;

(SEQ ID NO: 24)

R: 5′-TTCTCTACATATTTACTGCCAACA-3′;

(SEQ ID NO: 27)

F: 5′-TCTGTTCTCAGTTTTCCTGG-3′;

(SEQ ID NO: 28)

R: (5′-TCTTACCTCTTCTAGTTGGC-3′;

(SEQ ID NO: 31)

F: 5′-CCTTGGGCCAATAAGGTAAG-3′;

(SEQ ID NO: 32)

R: 5′-AGCTACTTGCAGTGTAACAGCATTT-3′;

(SEQ ID NO: 35)

F: 5′-CTGATTCATAGCAGCACTTG-3′;

(SEQ ID NO: 36)

R: 5′-AAAACATTTCCATTACCACTG-3′.

A variant oligonucleotide has, for example, at least 60% identity, preferably 80%, more preferably 95% identity or more, with the primer from which its sequence derives.

Said variant oligonucleotide has one or more sequence modifications with respect to the primer from which its sequence derives, in particular one or more deletion, addition or substitution type modifications. The added nucleotides are in particular added to the 3′ end.

In a specific example, said variant oligonucleotide has the same size as the primer from which its sequence derives.

In another specific example, the length of said variant oligonucleotide is shorter, for example by 10% to 20% with respect to that of the primer from which its sequence derives or possibly a greater size, for example by 10% to 40%, in particular 30% to 40% with respect to the sequence of the primer from which it derives.

Said oligonucleotide has the same properties as the primer from which it derives if it can be used as a primer to amplify DNA from a biological cell sample, in particular to amplify sequences comprising markers of fetal origin of a cell or to amplify sequences comprising genetic markers located upstream, downstream or on the CFTR gene characteristic of cystic fibrosis.

During step b) of the method of the invention, the cells retained on the filter may be observed under a microscope, for example after staining with hematoxylin and eosin to analyze their morphology in order to determine a presumption of their fetal origin. Their epithelial nature may be identified, for example, by immunolabeling with an anticytokeratin antibody (type KL₁). At this stage it is possible to envisage recognizing cells of fetal origin on the basis of morphological character, in particular cytotrophoblasts, mononuclear cells with a large nucleus, a condensed chromatin and a reduced cytoplasm, with a diameter in the range 14 to 20 μm, and syncytiotrophoblastic cells with a larger diameter (44-47 μm or more) and plurinuclear.

The term “presumption” or “presumed” in step b) thus indicates the strong probability of being in the presence of a cell of fetal origin.

Genetic analysis of cells retained during step a) can strengthen that presumption by obtaining indications as to the fetal or maternal origin of each of these cells. In particular, with a view to sensitive and specific detection or diagnosis, step a) will be repeated if no cell which is presumed to be of fetal origin is observed following it. Depending on the implementations, genetic analysis will be established on all of the cells retained during step a) and comprising certain cells the fetal origin of which is presumed, in particular on the genome of a small number of cells, from 1 to 20 cells, more particularly 1 to 10 cells, or in contrast analysis will be carried out, for example, on genomes from 1, 2 or 3 cells and advantageously on the genome of individually isolated cells.

In a particular implementation, analysis of a presumption of the fetal or maternal origin of cells retained on the filter is carried out by investigating the presence of immunological or cytological markers characteristic of fetal cells.

The term “immunological marker characteristic of fetal cells” means any antigen or combination of antigens the expression of which is normally significantly different between fetal cells and maternal cells, and which may be detected using an antibody or a combination of antibodies specifically directed against said antigen or combination of antigens. Particular examples of said immunological markers are the antigens associated with trophoblastic cells described in International patent application WO-A-90/06509. The disclosure of that application is hereby incorporated by reference into the present application. As an example, an investigation into the presence of immunological markers characteristic of fetal cells consists of:
bringing cells contained in the maternal sample into contact with at least one antibody directed against an antigen, which is characteristic of fetal cells; and
determining on cells retained on the filter a specific binding of said antibody with an antigen expressed on the surface of said cells; said contact of cells with the antibody being carried out before or after the filtration step. The selected antibodies may be polyclonal or monoclonal in type.

An example of antigen that is characteristic of fetal cells is the antigen of placentary alkaline phosphatase.

In another implementation, a presumption of the fetal origin of cells may be analyzed by determining specific cytological markers of cytotrophoblast and/or syncyciotrophoblastic cells. Cytological markers which may be used include all of the cytological characteristics of fetal cells which can differentiate them from other types of circulating cells which may be retained on the filter, in particular cell size, shape, the presence and size of particular organites, the nucleus size and number, chromatin structure, etc, or any particular combinations of these cytological characteristics. The cytological characters may be observed by staining the cells using stains which are conventionally used in cytology, in particular hematoxylin-eosin, and by observing labeled cells by optical microscopy.

The invention also pertains to a polynucleotide which can be used as a primer to amplify a quantity of DNA from a biological sample, characterized in that it comprises or consists of a

F: 5′-AAAAACCCTGGCTTATGC-3′;
R: 5′-AGCTACCATAGGGCTGGAGG-3′; or
F: 5′-GGAATCTGTTCTGGCAATGGAT-3′;
R: 5′-TTGCAATGAGCCGAGATCCTG-3′; or
F: 5′-CAGATGCTCGTTGTGCACAA-3′;
R: 5′-ATACCATTTACGTTTGTGTGTG-3′, or
F: 5′-TGACAGTGCAGCTCATGGTC-3′;
R: 5′-GGTCATTGGTCAAGGGCTGCT-3′; ou
F: 5′-TTGACATTCTTCTGTAAGGAAGA-3′;
R: 5′-AGGCTTGCCAAAGATATTAAAAGG-3′; or
F: 5′-TTGTGAATAGTGCTGCAATG-3′;
R: 5′-ATGTACACTGACTTGTTTGAG-3′; or
F: 5′-TGGAGCCTTCAGAGGGTAAA-3′;
R: 5′-TGCATAATCAAAAAGTTTTCACA-3′; or
F: 5′-TCTGTTCTCAGTTTTCCTGG-3′;
R: (5′-TCTTACCTCTTCTAGTTGGC-3′; or

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′.

A variant oligonucleotide has, for example, at least 60% identity, preferably 80%, more preferably 95% identity or more with the primer from which its sequence derives.

Said variant oligonucleotide has one or more sequence modifications with respect to the primer from which its sequence derives, in particular one or more deletion, addition or substitution type modifications.

In a specific example, said variant oligonucleotide is the same size as the primer from which its sequence derives.

In a further specific example, the length of said variant oligonucleotide is shorter by 10% to 20% with respect to that of the primer from which its sequence derives, or possibly larger, for example by 10% to 40%, in particular 30% to 40%, with respect to the sequence of the primer from which its sequence derives.

The variant primers of the oligonucleotides cited above may, for example, be derived from these oligonucleotides by addition of one or more nucleotides at the 5′ end of said sequences.

Said oligonucleotide has the same properties as the primer from which its sequence derives if it can be used as a primer to amplify DNA from a biological cell sample, in particular to amplify sequences comprising markers of the fetal origin of a cell, or to amplify sequences comprising a mutation of the F508 locus or sequences comprising genetic polymorphism markers forming a linkage with the CFTR gene characteristic of cystic fibrosis.

The invention also pertains to a pair of polynucleotides for use as a pair of primers to amplify a quantity of DNA from a biological sample, characterized in that it is selected from pairs of polynucleotides the sequences of which comprise or consist of:

F: 5′-AAAAACCCTGGCTTATGC-3′;
R: 5′-AGCTACCATAGGGCTGGAGG-3′;
F: 5′-GGAATCTGTTCTGGCAATGGAT-3′;
R: 5′-TTGCAATGAGCCGAGATCCTG-3′;
F: 5′-CAGATGCTCGTTGTGCACAA-3′;
R: 5′-ATACCATTTACGTTTGTGTGTG-3′;
F: 5′-TGACAGTGCAGCTCATGGTC-3′,
R: 5′-GGTCATTGGTCAAGGGCTGCT-3′;,
F: 5′-TTGACATTCTTCTGTAAGGAAGA-3′;
R: 5′-AGGCTTGCCAAAGATATTAAAAG-3′;
F: 5′-TTGTGAATAGTGCTGCAATG-3′;
R: 5′-ATGTACACTGACTTGTTTGAG-3′;
F: 5′-TGGAGCCTTGAGAGGGTAAA-3′;
R: 5′-TGCATAATCAAAAAGTTTTCACA-3′;
F: 5′-TCTGTTCTCAGTTTTCCTGG-3′;
R: (5′-TCTTACCTCTTCTAGTTGGC-3′;

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′.

The invention also pertains to an association of a pair of primers termed external primers with respect to the target sequence used in a first amplification phase of a DNA preparation and another pair of primers, termed internal or nested primers with respect to said external primers, used in a second amplification phase on the amplification product obtained by said first amplification phase, said pairs being selected from:

| | |
|---|---|
| F: 5′-AAAAACCCTGGCTTATGC-3′; | |
| R: 5′-AGCTACCATAGGGCTGGAGG-3′ | |
| as external primers; and | |
| F: 5′-CTTGGGGACTGAACCATCTT-3′; | |
| R: 5′-AGCTACCATAGGGCTGGAGG-3′ | |
| as internal primers; or | |
| F: 5′-GGAATCTGTTCTGGCAATGGAT-3′; | |
| R: 5′-TTGCAATGAGCCGAGATCCTG-3′ | |
| as external primers; and | |
| F: 5′-AAAGGCCAATGGTATATCCC-3′; | |
| R: 5′-GCCCAGGTGATTGATAGTGC-3′ | |
| as internal primers; or | |
| F: 5′-CAGATGCTCGTTGTGCACAA-3′; | |
| R: 5′-ATACCATTTACGTTTGTGTGTG-3′ | |
| as external primers; and | |
| F: 5′-GATCCCAAGCTCTTCCTCTT-3′; | |
| R: 5′-ACGTTTGTGTGTGCATCTGT-3′ | |
| as internal primers; or | |
| F: 5′-TGACAGTGCAGCTCATGGTC-3′ | |
| R: 5′-GGTCATTGGTCAAGGGCTGCT-3′; | |
| as external primers and | |
| F: 5′-GGATAAACATAGAGCGACAGTTC-3′; | |
| R: 5′-AGACAGAGTCCCAGGCATT-3′ | |
| as internal primers; or | |
| F: 5′-TTGACATTCTTCTGTAAGGAAGA-3′; | |
| R: 5′-AGGCTTGCCAAAGATATTAAAAG-3′ | |
| as external primers; and | |
| F: 5′-CCCTCTCAATTGTTTGTCTACC-3′; | |
| R: 5′-GCAAGAGATTTCAGTGCCAT-3′ | |
| as internal primers; or | |
| F: 5′-TTGTGAATAGTGCTGCAATG-3′; | |
| R: 5′-ATGTACACTGACTTGTTTGAG-3′ | |
| as external primers and | |
| F: 5′-ATGTACATGTGTCTGGGAAGG-3′; | |
| R: 5′-TTCTCTACATATTTACTGCCAACA-3′ | |
| as internal primers, or | |
| F: 5′-TGGAGCCTTCAGAGGGTAAA-3′; | |
| R: 5′-TGCATAATCAAAAAGTTTTCACA-3′ | |
| as external primers; and | |
| F: 5′-TCTGTTCTCAGTTTTCCTGG-3′; | |
| R: 5′-TCTTACCTCTTCTAGTTGGC-3′ | |
| as internal primers; or | |
| F: 5′-AAGTAATTCTCCTGCCTCAG-3′; | (SEQ ID NO: 29) |
| R: 5′-AGCTACTTGCAGTGTAACAGCATTT-3′ | (SEQ ID NO: 30) |
| as external primers and | |
| F: 5′-CCTTGGGCCAATAAGGTAAG-3′; | (SEQ ID NO: 31) |
| R: 5′-AGCTACTTGCAGTGTAACAGCATTT-3′ | (SEQ ID NO: 32) |
| as internal primers; or | |
| F: 5′-GAATTATAACCGTAACTGATTC-3′; | (SEQ ID NO: 33) |
| R: 5′-GAGATAATGCTTGTCTGACTTC-3′ | (SEQ ID NO: 34) |
| as external primers; and | |
| F: 5′-CTGATTCATAGCAGCACTTG-3′; | (SEQ ID NO: 35) |
| R: 5′-AAAACATTTCCATTACCACTG-3′ | (SEQ ID NO: 36) |
| as internal primers | |

The invention also concerns the use of primers in the context of an in vitro non-invasive prenatal detection method of the normal healthy condition, the healthy carrier condition or the condition of a carrier afflicted with cystic fibrosis from genomic DNA from fetal cells isolated from a maternal sample, characterized in that the primers are selected from:

F: 5′-AAAAACCCTGGCTTATGC3′;
R: 5′-AGCTACCATAGGGCTGGAGG-3′;
F: 5′-GGAATCTGTTCTGGCAATGGAT-3′;
R: 5′-TTGCAATGAGCCGAGATCCTG-3′;
F: 5′-CTTGGGGACTGAACCATCTT-3′;
R: 5′-AGCTACCATAGGGCTGGAGG-3′;
F: 5′-AAAGGCCAATGGTATATCCC-3′;
R: 5′-GCCCAGGTGATTGATAGTGC-3′;

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′;

(SEQ ID NO: 31)

F: 5′-CCTTGGGCCAATAAGGTAAG-3′;

(SEQ ID NO: 32)

R: 5′-AGCTACTTGCAGTGTAACAGCATTT-3′;

(SEQ ID NO: 35)

F: 5′-CTGATTCATAGCAGCACTTG-3′;

(SEQ ID NO: 36)

R: 5′-AAAACATTTCCATTACCACTG-3′;
F: 5′-TGGAGCCTTCAGAGGGTAAA-3′;
R: 5′-TGCATAATCAAAAAGTTTTCACA-3′;
F: 5′-TCTGTTCTCAGTTTTCCTGG-3′;
R: (5′-TCTTACCTCTTCTAGTTGGC-3′.

The invention also concerns the use of primers in the context of an in vitro method for identifying the fetal character of a single cell collected from a maternal sample, in a DNA preparation derived from the genome of the single collected cell, characterized in that the primers are selected from:

F: 5′-AAAAACCCTGGCTTATGC-3′;
R: 5′-AGCTACCATAGGGCTGGAGG-3′;
F: 5′-GGAATCTGTTCTGGCAATGGAT-3′;
R: 5′-TTGCAATGAGCCGAGATCCTG-3′;
F: 5′-CAGATGCTCGTTGTGCACAA-3′;
R: 5′-ATACCATTTACGTTTGTGTGTG-3′;
F: 5′-TGACAGTGCAGCTCATGGTC-3′;
R: 5′-GGTCATTGGTCAAGGGCTGCT-3′; ou
F: 5′-TTGACATTCTTCTGTAAGGAAGA-3′;
R: 5′-AGGCTTGCCAAAGATATTAAAAG-3′;
F: 5′-TTGTGAATAGTGCTGCAATG-3′;
R: 5′-ATGTACACTGACTTGTTTGAG-3′;

(SEQ ID NO: 29)

F: 5′-AAGTAATTCTCCTGCCTCAG-3′;

(SEQ ID NO: 30)

R: 5′-AGCTACTTGCAGTGTAACAGCATTT-3′;

(SEQ ID NO: 33)

F: 5′-GAATTATAACCGTAACTGATTC-3′;

(SEQ ID NO: 34)

R: 5′-GAGATAATGCTTGTCTGACTTC-3′;
F: 5′-CTTGGGGACTGAACCATCTT-3′;
R: 5′-AGCTACCATAGGGCTGGAGG-3′;
F: 5′-AAAGGCCAATGGTATATCCC-3′;
R: 5′-GCCCAGGTGATTGATAGTGC-3′;
F: 5′-GATCCCAAGCTCTTCCTCTT-3′;
R: 5′-ACGTTTGTGTGTGCATCTGT-3′;
F: 5′-GGATAAACATAGAGCGACAGTTC-3′;
R: 5′-AGACAGAGTCCCAGGCATT-3′;
F: 5′-CCCTCTCAATTGTTTGTCTACC-3′;
R: 5′-GCAAGAGATTTCAGTGCCAT-3′;
F: 5′-ATGTACATGTGTCTGGGAAGG-3′;
R: 5′-TTCTCTACATATTTACTGCCAACA-3′;

(SEQ ID NO: 31)

F: 5′-CCTTGGGCCAATAAGGTAAG-3′;

(SEQ ID NO: 32)

R: 5′-AGCTACTTGCAGTGTAACAGCATTT-3′;

(SEQ ID NO: 35)

F: 5′-CTGATTCATAGCAGCACTTG-3′;

(SEQ ID NO: 36)

R: 5′-AAAACATTTCCATTACCACTG-3′.

The invention also pertains to a kit for non-invasive prenatal detection of the normal healthy condition, the healthy carrier condition or the condition of a carrier afflicted with cystic fibrosis condition comprising one or more primers cited above, and if appropriate, instructions for detecting cystic fibrosis.

The means and methods (in particular primers) used in the context of the present invention may be used in the context of detection of cystic fibrosis and may if appropriate be considered as they are or supplemented by other tests as diagnostic means and methods.

### KEY TO FIGURES

FIG. 1: Prenatal non-invasive diagnosis in couples carrying the ΔF508 allele

Genotyping by STR in couple 1 (A) with the D16S539 marker and couple 6 (A′) with the D16S3018 marker. In couple 1 (A), the father (P) was homozygous for the target allele, while the mother (M) was heterozygous. Two fetal cells (CF) are shown, characterized by a paternal allele (left) and a maternal allele (right). In couple 6 (A′), the two parents were heterozygous with the STR marker used and the two fetal cells (CF) had a maternal allele (left) and a paternal allele (right). Genotyping of ΔF508 (B and B′) carried out on the fetal cells of couple 1 (B) and 6 (B′) showed that the fetus from couple 1 was a carrier of the ΔF508 allele having a mutated allele (AM) and a normal allele (AN), while the fetus of couple 6 was completely normal (homozygous for normal allele). C and C′: sequencing of the ΔF508 locus in fetal cells confirmed the diagnosis by showing both the mutated profile and normal profile in the fetus of couple 1 (C), and the homozygous presence of the normal allele in the fetus of couple 6 (C′).

FIG. 2: Non-invasive prenatal diagnosis in couple no 11
A. DNA genotyping with the informative marker D7S486 in chromosome 7, showing that the parents (P and M) were heterozygous and the afflicted child (CI: index case) carried mutated alleles d) and a). The two fetal cells (CF) of the pregnancy carry a single mutated allele (d), demonstrating that the fetus was a carrier but not afflicted.
B. DNA genotyping of ΔF508 locus, showing that the father was a carrier of a mutated allele (AM) and a normal allele (AN). The index case is a carrier of the mutated ΔF508 allele, while the two fetal cells from pregnancy are homozygous for the normal allele. Hence, the fetus is a carrier of a mutation on chromosome 7 which is not the ΔF508 mutation.
C. Diagrammatic representation of two chromosomes 7 in paternal DNA (P), maternal DNA (M), the index case (CI) and the circulating fetal cells (CF): the father carries a ΔF508 allele while the mother carries an unknown mutation. The index case carries the two mutations and the fetal cells carry only the unknown mutation.

FIG. 3: Non-invasive prenatal diagnostic in couple no 11
A. DNA genotyping with the informative marker D7S486 in chromosome 7, showing that the parents (P and M) are heterozygous and the index case (CI) carries mutated alleles d) and a). The two fetal cells (CF) of the pregnancy carry non mutated alleles (c and b), demonstrating that the fetus is completely normal.
B. Diagrammatic representation of two 7 chromosomes in paternal DNA (P), maternal DNA (M), the index case (CI) and the circulating fetal cells (CF): the father carries a mutated allele a), while the mother carries the mutated allele d). The afflicted child carries both mutated alleles and the two circulating fetal cells carry the two normal alleles.

FIG. 4: Protocol for non-invasive prenatal diagnosis (NI-PND) of cystic fibrosis

STR (short tandem repeat);

ISET (isolation by size of epithelian tumor);

LCM (laser capture microdissection;

PEP (primer extension pre-amplification);

MC (maternal cells);

CFC (circulating fetal cells).

FIG. 5: Non-invasive prenatal diagnosis in couples carrying the ΔF508 allele

A, B, C: STR genotyping in couple 1 with the marker D16S539 (A), in couple 6 with the marker D16S3018 (B) and in couple 8 with the marker D21S1435 (C).

In couple (A), the father (P) is homozygous for the target allele (one peak), while the mother (M) is heterozygous (two peaks). Two circulating fetal cells (CFC) from this couple were characterized by a paternal allele (left) and a maternal allele (right). In couple 6 (B), and couple 8 (C), the two parents were heterozygous with the STR marker used and the two fetal cells (CFC) have one maternal allele (left) and one paternal allele (right).

A′, B′, C′: ΔF508 genotyping carried out on circulating fetal cells (CFC) from couple 1 (A′), couple 6 (B′) and couple 8 (c′) show that the fetus from couple 1 is a carrier of the ΔF508 allele, having one mutated allele (AM) and one normal allele (AN), while the fetus from couple 6 is completely normal (homozygous for normal allele) and the fetus from couple 8 is afflicted with cystic fibrosis (CF) (homozygous for mutated allele).

A″, B″, C″: sequencing of F508 locus in fetal cells from couple 1 (A″), couple 6 (B″) and couple 8 (C″) confirms the diagnosis by showing both the mutated and normal profile in the fetus from couple 1 (A″), and the homozygous presence of the normal allele in the fetus from couple 6 (B″) and the homozygous presence of the mutated allele in the fetus from couple 8 (C″).

FIG. 6: Non-invasive prenatal diagnosis of cystic fibrosis (CF) in couples with unknown CFTR mutations

A, B, C: DNA genotyping of couple 11 and their child afflicted with the informative marker D7S486 on chromosome 7, showing that the father (P) and mother (M) are heterozygous. The father carries the c) and a) alleles while the mother carries alleles d) and b). Their afflicted child (CI: index case) carries alleles d) and a) which must thus be linked to mutated CFTR alleles. Two CFCs from the pregnancy carry alleles d and c, demonstrating that the fetus is a carrier of cystic fibrosis but is not afflicted.

B: DNA genotyping of F508 locus showing that the father carries a mutated allele (AM) and a normal allele (AN). The index case presents a composed heterozygosity and carries the paternal ΔF508 allele and a maternal CFTR mutation. Two fetal cells are homozygous for the normal F508 allele. Taken together, the data from A and B show that the fetus is a carrier of the maternal CFTR mutation: diagrammatic representation of CFTR alleles and STR alleles linked to CFTR (a, b, c, d). Gray block=unknown CFTR mutation.

D, E: STR genotyping of DNA from couple 12 with the D7S486 marker on chromosome 7 shows that the father and mother are heterozygous and that the index case carries the a) and d) D7S486 alleles, which must thus be linked to the mutated CFTR alleles. Two CFCs from the pregnancy carry the c) and d) D7S486 alleles linked to mutated CFTR alleles, demonstrating that the fetus is completely normal.

E: Diagrammatic representation of CFTR alleles and STR alleles linked to CFTR (a, b, c, d). Gray blocks=mutated CFTR alleles.

### EXAMPLES

### Example 1

### Non-Invasive Diagnosis of Predisposition to Cystic Fibrosis by Effective Enrichment of Trophoblastic Cells and Analysis of Mutations Limited to Genomes of Single Cells shown to be Fetal by Genotyping

### 1-1 Method

Peripheral blood from 12 women in weeks 11 to 13 weeks of pregnancy necessitating a prenatal diagnosis of cystic fibrosis were studied by ISET (isolation by size of tumoral epithelial/trophoblastic cells).

Six ml of maternal blood (before CVS) and 2 ml of paternal blood were collected on an ethylenediaminetetraacetic acid buffer. The paternal and maternal DNA were extracted from 1 ml of blood and 1.5 ng was used for allelotyping with specific primers of micro-satellite markers (STR) D7S480, D7S486, D16539, D16S3018, D21S1435 and D21S1437. A set of external (out) primers and internal (in) primers (fluorescein-containing) was used for each STR marker (see Table 1 for the sequence of primers and the PCR profiles). Four ml of maternal blood was treated by ISET until 3 hours following collection, as described above (Vona et al, 2000, 2002). Briefly, the blood samples were diluted to 1:10 with the ISET buffer and treated with the ISET apparatus (Metagenex, Paris, France). The large cells from each ml of blood were concentrated on a 0.6 cm diameter circular spot on the ISET membrane. Two ml of each maternal blood sample treated by ISET was analyzed. After immunohistochemical analysis with the KL1 antibody (Vona et al, 2002) to identify epithelial cells and staining with hematoxylin, laser micro-dissection with a laser of a single cell was carried out using a Leica (AS LMD) and a microscope equipped with a Nikon laser (TE 2000 U, Nikon France SA). To ensure that a single cell had been collected, we took photographs of the cell before and after micro-dissection and of the microdissected cell on the clot.

Each collected cell was lyzed in 15 μl of lysis buffer (Tris-HCl 100 mmol/l, ph 8, proteinase K 400 μg/ml) for 16 hours at 37° C., followed by deactivating the proteinase K at 94° C. for 15 minutes. After primer extension pre-amplification (PEP) carried out as described above (Vona et al, 2000, 2002) in a volume of 60 μl, we used 6 μl aliquots for the supplemental amplifications. Genotyping of single cells was carried out using specific STR primers presented as informatives by genotyping the maternal and paternal DNA. Another aliquot of 6 μl of PEP product obtained from single cells and shown to be fetal by STR genotyping was used to investigate the ΔF508 allele using primers comprising the ΔF508 locus. Amplification was carried out in 40 μl containing 6 μl of PEP product, 10 mmol/l of Tris-HCl, 50 mmol/l of KCl, 25 mmol/l of MgCl₂, 200 μmol/l of each deoxynucleotide, 0.5 μM of each external primer and 2 U of Taq Gold (Applied Biosystems, Foster City, Calif.). 2 μl of the first PCR product was again amplified in 40 μl using internal primers and the same protocol (see Table 1 for the primer sequence and PCR profiles). 1 μl of PCR product diluted to 1:20 was then mixed with 13.5 μl of deionized Hi-Di formamide, 0.5 μl of Genescan 400 HD marker (ROX) (Applied Biosystems, Foster City, Calif.) and loaded onto an ABI Prism 3100 automatic sequencer (Applied Biosystems, Foster City, Calif.). The profiles were analyzed using Genescan and Genotyper software (Perkin Elmer, Foster City, Calif.).

TABLE 1
Primers and PCR profiles

| | | | |
|---|---|---|---|
| Name of primer | Sequence number | Primer number | PCR profiles |

| | | | |
|---|---|---|---|
| D7S480 out* | SEQ ID NO: 1 | F: 5′-AAAAACCCTGGCTTATGC-3′ | 5 min 94° C., 40 × (30 s 94° C., 30 s |
| | SEQ ID NO: 2 | R: 5′-AGCTACCATAGGGCTGGAGG-3′ | 58° C., 30 s 72° C.), 5 min 72° C. |
| D7S480 in* | SEQ ID NO: 3 | F: 5′-CTTGGGGACTGAACCATCTT-3′ | 5 min 94° C., 40 × (30 s 94° C., 45 s |
| | SEQ ID NO: 4 | R: 5′-AGCTACCATAGGGCTGGAGG-3′ | 55° C., 30 s 72° C.), 5 min 72° C. |
| D7S486 out* | SEQ ID NO: 5 | F: 5′-GGAATCTGTTCTGGCAATGGAT-3′ | 5 min 94° C., 40 × (30 s 94° C., 45 s |
| | SEQ ID NO: 6 | R: 5′-TTGCAATGAGCCGAGATCCTG-3′ | 55° C., 30 s 72° C.), 5 min 72° C. |
| D7S486 in* | SEQ ID NO: 7 | F: 5′-AAAGGCCAATGGTATATCCC-3′ | 5 min 94° C., 40 × (30 s 94° C., 30 s |
| | SEQ ID NO: 8 | R: 5′GCCCAGGTGATTGATAGTGC-3′ | 55° C., 30 s 72° C.), 5 min 72° C. |
| D16S539 out | SEQ ID NO: 9 | F: 5′-CAGATGCTCGTTGTGCACAA-3′ | 5 min 94° C., 40 × (30 s 94° C., 45 s |
| | SEQ ID NO: 10 | R: 5′-ATACCATTTACGTTTGTGTGTG-3′ | 60° C., 30 s 72° C.), 5 min 72° C. |
| D16S539 in | SEQ ID NO: 11 | F: 5′-GATCCCAAGCTCTTCCTCTT-3′ | 5 min 94° C., 40 × (30 s 94° C., 30 s |
| | SEQ ID NO: 12 | R: 5′-ACGTTTGTGTGTGCATCTGT-3′ | 58° C., 30 s 72° C.), 5 min 72° C. |
| D16S3018 out | SEQ ID NO: 13 | F: 5′-TGACAGTGCAGCTCATGGTC-3′ | 5 min 94° C., 40 × (30 s 94° C., 30 s |
| | SEQ ID NO: 14 | R: 5′-GGTCATTGGTCAAGGGCTGCT-3′ | 61° C., 30 s 72° C.), 5 min 72° C. |
| D16S3018 in | SEQ ID NO: 15 | F: 5′-GGATAAACATAGAGCGACAGTTC-3′ | 5 min 94° C., 40 × (30 s 94° C., 30 s |
| | SEQ ID NO: 16 | R: 5′-AGACAGAGTCCCAGGCATT-3′ | 58° C., 30 s 72° C.), 5 min 72° C. |
| D21S1435 out | SEQ ID NO: 17 | F: 5′-TTGACATTCTTCTGTAAGGAAGA-3′ | 5 min 94° C., 40 × (30 s 94° C., 30 s |
| | SEQ ID NO: 18 | R: 5′-AGGCTTGCCAAAGATATTAAAAG-3′ | 58° C., 42 s 72° C.), 5 min 72° C. |
| D21S1435 in | SEQ ID NO: 19 | F: 5′-CCCTCTCAATTGTTTGTCTACC-3′ | 5 min 94° C., 40 × (30 s 94° C., 30 s |
| | SEQ ID NO: 20 | R: 5′GCAAGAGATTTGAGTGCCAT-3′ | 58° C., 30 s 72° C.), 5 min 72° C. |
| D21S1437 out | SEQ ID NO: 21 | F: 5′-TTGTGAATAGTGCTGCAATG-3′ | 5 min 94° C., 40 × (30 s 94° C., 45 s |
| | SEQ ID NO: 22 | R: 5′-ATGTACACTGACTTGTTTGAG-3′ | 60° C., 30 s 72° C.), 5 min 72° C. |
| D21S1437 in | SEQ ID NO: 23 | F: 5′-ATGTACATGTGTCTGGGAAGG-3′ | 5 min 94° C., 40 × (30 s 94° C., 45 s |
| | SEQ ID NO: 24 | R: 5′-TTCTCTACATATTTACTGCCAACA-3′ | 58° C., 30 s 72° C.), 5 min 72° C. |
| Delta F508 out | SEQ ID NO: 25 | F: 5′-TGGAGCCTTCAGAGGGTAAA-3′ | 5 min 94° C., 40 × (30 s 94° C., 30 s |
| | SEQ ID NO: 26 | R: 5′-TGCATAATCAAAAAG TTT TCACA-3′ | 55° C., 30 s 72° C.), 5 min 72° C. |
| Delta F508 in | SEQ ID NO: 27 | F: 5′-TCT GTT CTCAGT TTT CCTGG-3′ | 5 min 94° C., 40 × (30 s 94° C., 30 s |
| | SEQ ID NO: 28 | R: 5′-TCT TAC CTC TTC TAG TTG GC-3′ | 57° C., 30 s 72° C.), 5 min 72° C. |
| D7S490 out | SEQ ID NO: 29 | F: 5′-AAGTAATTCTCCTGCCTCAG-3′ | 5 min 94° C., 40 × (30 s 94° C., 30 s |
| | SEQ ID NO: 30 | R: 5′-AGCTACTTGCAGTGTAACAGCATTT-3′ | 58° C., 30 s 72° C.), 5 min 72° C. |
| D7S490 in | SEQ ID NO: 31 | F: 5′-CCTTGGGCCAATAAGGTAAG-3′ | 5 min 94° C., 40 × (30 s 94° C., 30 s |
| | SEQ ID NO: 32 | R: 5′-AGCTACTTGCAGTGTAACAGCATTT-3′ | 55° C., 30 s 72° C.), 5 min 72° C. |
| D7S523 out | SEQ ID NO: 33 | F: 5′-GAATTATAACCGTAACTGATTC-3′ | 5 min 94° C., 40 × (30 s 94° C., 30 s |
| | SEQ ID NO: 34 | R: 5′-GAGATAATGCTTGTCTGACTTC-3′ | 58° C., 30 s 72° C.), 5 min 72° C. |
| D7S523 in | SEQ ID NO: 35 | F: 5′-CTGATTCATAGCAGCACTTG-3′ | 5 min 94° C., 40 × (30 s 94° C., 30 s |
| | SEQ ID NO: 36 | R: 5′-AAAACATTTCCATTACCACTG-3′ | 58° C., 30 s 72° C.), 5 min 72° C. |

*Primers used for indirect diagnosis.

### 2-2 Results

In couples in whom none or only one of the parents was a carrier of the ΔF508 allele, we requested information from the Laboratoire de Genetique Medicale [Medical Genetics Laboratory] regarding a first afflicted child (index case) and informative STR primers located on chromosome 7. We then used those primers to amplify DNA from fetal cells and carried out an indirect diagnostic method (see FIGS. 2 and 3). The sequences were analyzed using a Big Dye Terminator sequencing kit (Applied Biosystems, Foster City, Calif.) (Vona et al, 2002) after purification of the PCR product on Microspin S-400RH columns (Amersham Bioscience, Buckinghamshire, GB). Blood samples from the mothers in couples 4 and 12 (Table 2) were analyzed using a completely blind approach, by researchers ignorant of the CVS results.

At least two informative markers (Table 2) identified from among those tested on the maternal and paternal DNA isolated from PBL cells (Table 1) were used for allelotyping micro-dissected cells. This allowed us to identify at least two fetal cells from only 2 ml of maternal blood.

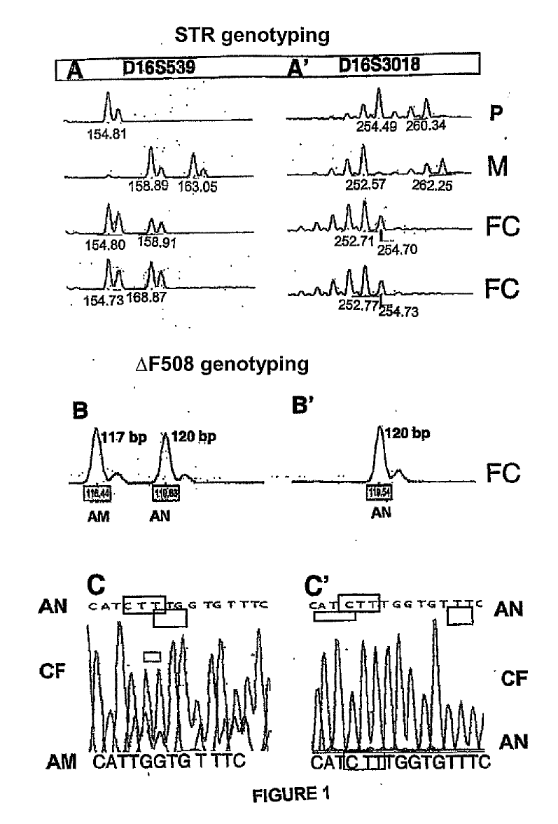

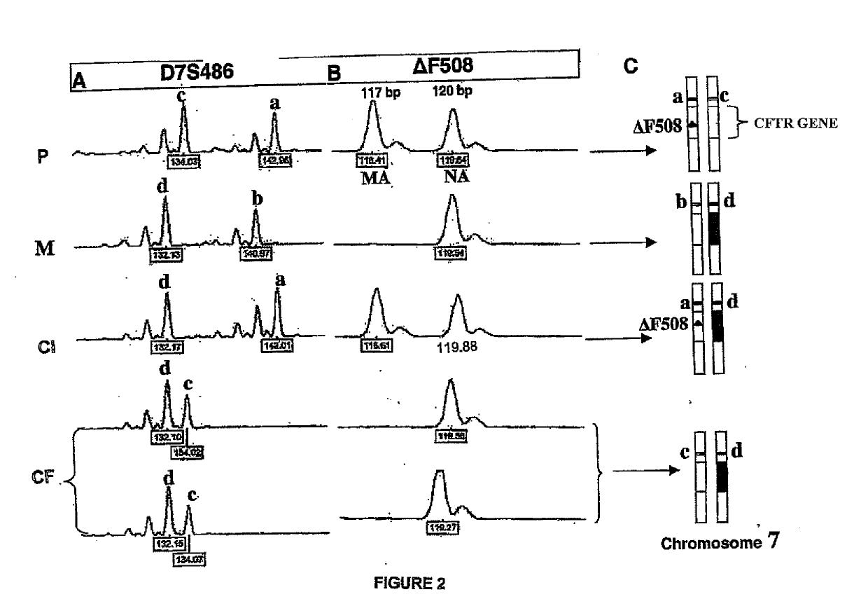

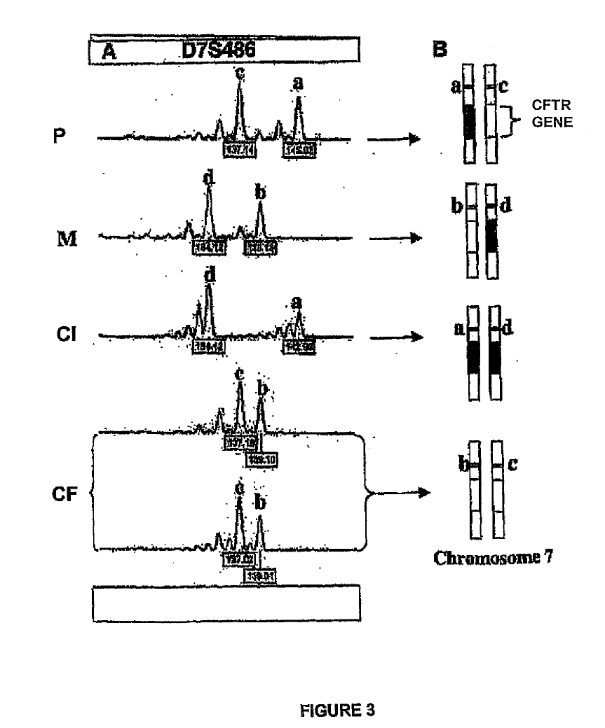

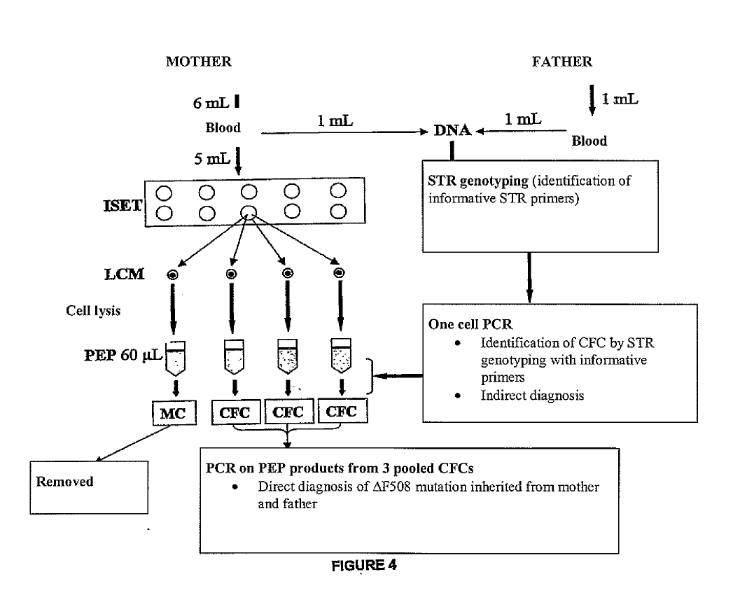

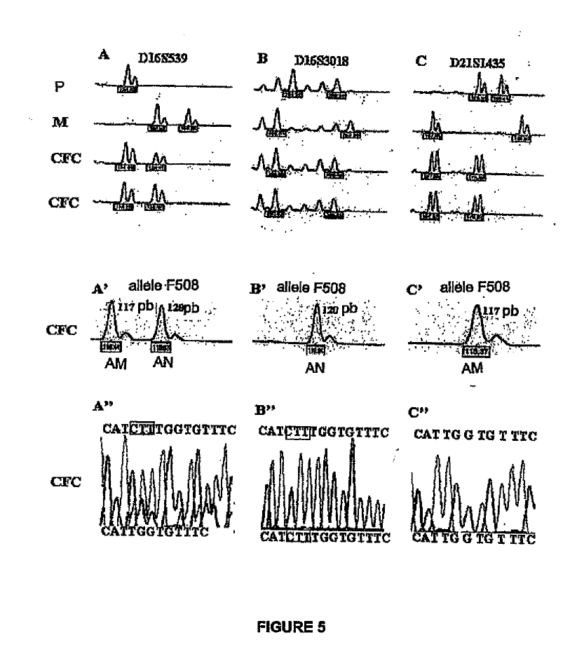

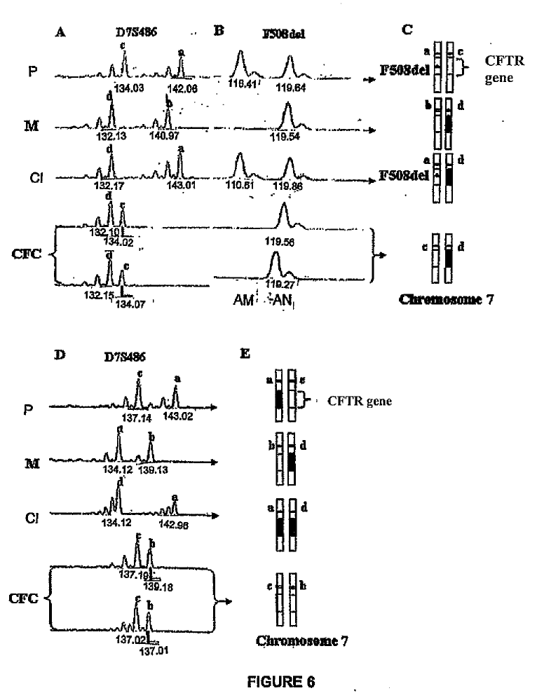

The DNA from single fetal cells was then analyzed with nested primers comprising the ΔF508 locus. This analysis could reveal the ΔF508 allele which is characterized by a 3 bp (CTT) deletion (117 bp PCR product instead of 120 bp, see FIG. 1), allowing us to discern whether the fetus was afflicted with cystic fibrosis (homozygous presence of ΔF508 allele), a carrier of the ΔF508 allele (heterozygous presence of ΔF508 allele) or normal (homozygous absence of ΔF508 allele). As can be seen in Table 2, the heterozygous presence of the ΔF508 allele was discovered in all of the fetal cells isolated from maternal blood from 7 women from couples carrying the ΔF508 allele. In 6 fetal cells, this result was confirmed by sequencing the specific PCR product of ΔF508 (FIG. 1). Fetal cells isolated from the maternal blood from 3 other couples carrying the ΔF508 allele were shown to be homozygous for the absence of the ΔF508 allele. Fetal cells isolated from couple 11 (FIG. 2) showed the homozygous absence of the ΔF508 allele. The presence of an unknown mutation in the paternal allele was investigated by informative STR primers on chromosome 7 (indirect diagnosis). The analysis proved the presence of the mutated allele in the analyzed fetal cells. Fetal cells isolated from the maternal blood of couple 12 were tested with informative STR primers on chromosome 7 (see Table 2) to produce an indirect diagnosis. The results showed that the fetus had inherited non mutated alleles both from the mother and the father (FIG. 3 and Table 2). All of the results we obtained on the fetal cells were coherent with the results obtained by CVS.

TABLE 2
Prenatal diagnosis of cystic fibrosis on circulating fetal cells (non-invasive method)

| | | No of fetal | Prenatal diagnostic | |
|---|---|---|---|---|
| Couple | Informative markers | cells tested | Invasive (CVS) | Non-invasive (ISET) |
| 1 | D7S486/D16S539 | 3 | Hétérozygote Δ F508 | Hétérozygote Δ F508 |
| 2 | D16S539/D21S1435 | 2 | Homozygote NL | Homozygote NL |
| 3 | D7S486/D16S3018 | 2 | Hétérozygote Δ F508 | Hétérozygote Δ F508 |
| 4 | D16S3018/D21S1435 | 1 | Homozygote NL | Homozygote NL |
| 5 | D16S3018/D21S1437 | 2 | Hétérozygote Δ F508 | Hétérozygote Δ F508 |
| 6 | D16S539/D16S3018 | 2 | Homozygote NL | Homozygote NL |
| 7 | D16S539/D21S1437 | 1 | Hétérozygote Δ F508 | Hétérozygote Δ F508 |
| 8 | D16S3018/D21S1435 | 1 | Hétérozygote Δ F508 | Hétérozygote Δ F508 |
| 9 | D16S539/D16S3018 | 3 | Hétérozygote Δ F508 | Hétérozygote Δ F508 |
| 10 | D7S486/D16S3018 | 2 | Hétérozygote Δ F508 | Hétérozygote Δ F508 |
| 11 | D7S486/D16S3018/D21S1435 | 2 | Hétérozygote MU² | Hétérozygote MU² |
| 12 | D7S480/D7S486/D16S3018 | 2 | Homozygote NL² | Homozygote NL² |

²Indirect diagnostic: CVS: removal of choral villosities; NL: normal; MU: unknown mutation

### 3-3 Discussion

We show here the feasibility of a non-invasive prenatal diagnosis of cystic fibrosis by effective enrichment of trophoblastic cells and analysis of mutations limited to the genomes of single cells shown to be fetal by genotyping. The results show that this approach is valid both for couples carrying the ΔF508 allele and for carriers of unknown mutations, provided that in the latter case information on a first afflicted child (index case) is available. From a technical viewpoint, application of the analysis of single cells to a prenatal diagnosis is limited by the risk of allele drop-out (ADO), the incapacity of PCR to amplify one of the two allele sequences. Our tests have confirmed that ADO is strictly linked to the sequence of the primers and the PCR profile. Once these parameters have been defined, ADO is no longer a limitation to our assay. During the single cell genotyping step, even if an ADO occurs (we have observed it twice), the only consequence is the loss of one fetal cell, without risking making an erroneous diagnosis. We have in fact confirmed the fetal genotype of the whole micro-dissected cell by two STR markers. To amplify the cystic fibrosis mutation in single fetal cells, we have obtained coherent results in the fetal cells analyzed. At this stage, it is possible to mix the PEP products obtained from two or more fetal cells, a procedure which is known to eliminate the risk of ADO. However, in this work, the results were obtained for single fetal cells without having to mix the DNA of several single fetal cells. The data described here show for the first time a reliable approach to carrying out a prenatal diagnosis of cystic fibrosis on circulating fetal cells, offering a prenatal choice to parents at risk of having a child afflicted with cystic fibrosis.

### Example 2

### Filtration of a Sample of Pure or Diluted Maternal Blood, to Concentrate on a Filter According to Size Certain Circulating Cells, in Particular Cells of Fetal Origin and Analysis of Cells Retained on the Filter to Obtain a Presumption of their Fetal or Maternal Origin

Blood samples were diluted 10 times in a filtration buffer containing 0.175% of saponin, 0.2% of para formaldehyde, 0.0372% of EDTA and 0.1% of BSA, then filtered using a calibrated polycarbonate filter with calibrated 8 μm diameter pores. Cells retained on the filter were collected on a circular spot 0.6 cm in diameter. After staining with eosin and hematoxylin, the spots were analyzed under a microscope and each cell was photographed under low and high magnification. The cell size was determined using Adobe Photoshop software, taking the 8 μm size of the pores as the reference. The photos allowed the cells to be identified under the Pixcell II Arcturus (Mountain View, Calif.) microscope. FIG. 2 shows the microscopic analysis obtained by this method.

Each cell was micro-dissected by laser capture without any pre-treatment of the filter. To ensure that a single cell was collected each time, the filter was photographed before and after micro-dissection, along with the micro-dissected cell deposited on the cap (CapSure™ HS). The cell was then lysed in 15 μl of lysis buffer (100 mM of Tris-HCl, pH 8, 400 μg/ml of proteinase K) for 16 hours at 37° C. The lysate was collected after centrifuging and the proteinase K was deactivated at 90° C. for 10 minutes. After pre-amplification by primer extension as described by Zhang et al (see above), the DNA was precipitated with ethanol and re-suspended in 10 μl of water. Each sample was then tested, firstly with the primers described in the present application.

### Example 3

### Amplification Steps

PCRs were carried out on a 20 μl volume of reaction mixture containing 2 μl of PEP product (primer extension pre-amplification), 10 mM of Tris-HCl, 50 mM of KCl, 1.5 mM of MgCl₂, 0.01% of gelatin, 200 mM of each deoxynucleotide, 20 picomoles of each primer used and 1 U of Taq polymerase (Perkin-Elmer Cetus, Emeryville, Calif.). After an initial denaturing step at 94° C. for 5 minutes, 40 amplification cycles were carried out (94° C. 30 s, 55° C. to 61° C. 30 to 45 s, 72° C. 30 s) using external primers of the invention and this amplified product was again amplified using internal primers of the invention and employing the same protocol, then a final elongation step was carried out at 72° C. for 5 minutes in a Perkin Elmer 9700 thermocycler (see Table 1).

### Example 4

### Detection of Mutations in the CFTR Gene Inherited from Father and Mother in Fetal Cells Isolated from Maternal Blood, Allowing a Safe Prenatal Diagnosis of Cystic Fibrosis

A multi-STR marker approach was developed to speed up the genotyping step on a single cell, and analysis of the ΔF508 mutation was carried out on genomes from three trophoblastic cells taken together (pooled genomes) to avoid problems with allele drop-out (ADO).

### 1.1 Method and Apparatus

Removal of Blood and ISET

Six ml of maternal blood (before CVS) and 1 ml of paternal blood were collected on an ethylenediaminetetraacetic acid (EDTA) buffer. The paternal and maternal DNA were extracted from 1 ml of blood and 1.5 ng was used for allele typing with primers which carried a fluorescein marker, and which were specific to the STR markers linked to the CFTR locus (D7S480, D7S486, D7S490, D7S523) or to other genomic loci (D16539, D16S3018, D21S1435 and D21S1437, see Table 1 above).

The remaining 5 ml of maternal blood was treated by ISET until 3 hours after removal, as described above, using the ISET apparatus (Metagenex, Paris, France; www.metagenex.fr) and stored at −20° C. (Beroud et al, 2003; Vona et al, 2002). Three ml of each maternal blood sample treated by ISET (i.e. three spots on the filter) were analyzed.

After immunohistochemical analysis with KL1 antibody (Vona et al, 2002) to identify epithelial cells, single cell laser micro-dissection was carried out using a microscope provided with a Nikon TE 2000 U laser (Nikon, Paris, France and MMI, Zurich, Switzerland). ISET enriches blood in epithelial cells but also retains about 0.02% of peripheral blood leukocytes (Vona et al, 2002; Vona et al, 2000).

Thus, to develop the molecular protocol for the non-invasive prenatal diagnosis of cystic fibrosis, blood from 5 known carriers of ΔF508 was treated by ISET and 75 single leukocytes cells were micro-dissected.

Cell Lysis, Primer Elongation Pre-Amplification and STR Genotyping.

Each micro-dissected cell was lyzed in 15 μl of lysis buffer (Tris-HCl 100 mmol/l, ph 8, proteinase K 400 μg/ml) for 2 hours at 60° C., followed by deactivation of the proteinase K at 94° C. for 15 minutes. After primer extension pre-amplification (PEP) (Zhang et al, 1992), 5 μl of a solution of 400 μM of random primers (genPEP™ 75 OD kit, Genetix, Boston, USA), 6 μl of PCR buffer (25 mM MgCl₂/gelatin (1 mg/ml), 100 mM of Tris-HCl, pH 8.3, 500 mM of KCl), 3 μl of a mixture of 4 dNTPs (each 2 mM) and 1 μl (5 U) of Taq polymerase (Applied Biosystems, foster City, USA) in a final volume of 60 μl were added to the lysed cell.

The other STR amplifications (see Table 1 above) were carried out in 40 μl containing 4 μl of PEP product, 10 mM of Tris-HCl, 50 mM of KCl, 2.5 mM of MgCl₂, 200 μM of each deoxynucleotide, 0.5 μM of each primer and 2 U of Taq Gold (Applied Biosystems, Foster City, Calif., USA). One μl of the PCR product diluted to 1:20 was then mixed with 13.5 μl of deionized Hi-Di formamide and 0.5 μl of Genescan 400 HD (ROX) marker (Applied Biosystems) and placed in an ABI Prism 3100 automatic sequencer (Applied Biosystems). The profiles were analyzed using Genescan and Genotyper programs (Applied Biosystems).

We tested the individual cell genotyping efficiency by calculating the PCR failure rate and allele drop-out (ADO) in STR tests carried out on 75 micro-dissected leukocytes. We used three heterozygous markers (see Table 3 above), giving two distinguishable alleles. The PCR efficiency was calculated as the number of cells which produce a PCR product, and the PCR accuracy was calculated as the number of cells which exhibit two alleles with at least one of the three STR markers used.

TABLE 3
PCR efficiency and amplification accuracy (AA) using STR- and F508-specific primers

| | No of tests | Primers | PCR efficiency | p value | AA (%) | ADO (%) | p value |
|---|---|---|---|---|---|---|---|
| Individual cell | 75 | F508 | 67 (90%) | | 61 (91%) | 6 (9%) | |
| PCR | | | | | | | |
| Group PCR^{a} | 30 | F508 | 30 (100%) | <0.001^{b} | 30 (100%) | 0 (0%) | <0.001^{c} |
| Individual cell | 75 | M1 | 58 (77%) | 0.1^{d} | 42 (72%) | 16 (28%) | 0.2^{d} |
| PCR | | M2 | 54 (72%) | 0.9^{e} | 38 (70%) | 16 (30%) | 0.9^{e} |
| | | M3 | 52 (69%) | 0.6^{f} | 39 (75%) | 13 (25%) | 0.4^{f} |
| | | M1 + M2 + M3^{g} | 71 (95%) | <0.001^{def} | 67 (94%) | 4 (6%) | <0.001^{def} |

Abbreviations:
STR, short tandem repeat;
AA: amplification accuracy;
ADO (allele drop-out);
M1: D16S3018;
M2: D16S539;
M3: D21S1435.
^{a}pooling of 3 PEP products from genomes of individual amplifiable cells;
^{b}vs PCR F508 efficiency for individual cell;
^{c}vs PCR F508 efficiency for individual cell;
^{d}vs M2;
^{e}vs M3;
^{f}vs M1;
^{g}markers tested for independent amplifications.

Protocol for Amplification of F508 Locus on Individual Cell

4 μl aliquots were removed from the 60 μl of PEP product for PCR amplifications using fluorescein-containing primers covering the F508 locus (see Table 1 above). The genomes of individual cells were tested individually (4 μl of PEP product per amplification), and in groups of three, by mixing 30 μl of PEP product and taking 12 μl (from the 90 μl) for F508 amplification. The amplifications were carried out in 40 μl containing 4 μl of PEP product (or 100 μl containing 12 μl of PEP product), 10 mM of Tris-HCl, 50 mM of KCl, 2.5 mM of MgCl₂, 200 μl of each deoxynucleotide, 0.5 μl of each primer and 2 U of Taq Gold (Applied Biosystems, Foster City, Calif., USA). This protocol avoided DNA purification steps which run a high risk of losing copies of DNA. 1 μl of PCR product diluted 1:20 was then mixed with 13.5 μl of deionized Hi-Ti formamide and 0.5 μl of Genescan 400 HT markers (ROX) (Applied Biosystems) and placed in an automatic ABI Prism 3100 sequencer (Applied Biosystems). The profiles were analyzed using Genescan and Genotyper programs (Applied Biosystems).

Application of Protocol to Non-Invasive Prenatal Diagnosis of Cystic Fibrosis in 12 Pregnant Women

We tested the peripheral blood of 12 women in the 11^{th} to 13^{th} week of pregnancy who had requested a prenatal diagnosis of cystic fibrosis, including two women who already had a child presenting composed heterozygosity for cystic fibrosis (1 index case; couples 11 and 12, Table 4). All of the women gave their informed consent to this study, which had been approved by the local ethics committee.

TABLE 4
Prenatal diagnosis of cystic fibrosis (CF) by genetic analysis of circulating fetal cells and by chorionic villus sampling

| | | | CF genotype on | CF genotype on | | |
|---|---|---|---|---|---|---|
| | | Number of | individual | groups of 3 | CF genotype by | |
| Pair | Informative STR markers | CFCs tested | CFCs^{b} | CFCs^{c} | CVS | Fetal condition |
| 1^{a} | D7S486/D16S539/D21S1435 | 4 | ΔF508/N | ΔF508/N | ΔF508/N | CF carrier |
| 2^{a} | D16S539/D21S1435/D21S1437 | 3 | N/N | N/N | N/N | Healthy |
| 3^{a} | D7S486/D16S3018/D21S1437 | 3 | ΔF508/N | ΔF508/N | ΔF508/N | CF carrier |
| 4^{a} | D16S539/D16S3018/D21S1435 | 4 | N/N | N/N | N/N | Healthy |
| 5^{a} | D16S3018/D21S1437/D21S1435 | 3 | ΔF508/N | ΔF508/N | ΔF508/N | CF carrier |
| 6^{a} | D7S480/D16S539/D16S3018 | 3 | N/N | N/N | N/N | Healthy |
| 7^{a} | D16S539/D21S1437/D21S1435 | 4 | ΔF508/N | ΔF508/N | ΔF508/N | CF carrier |
| 8^{a} | D7S480/D16S3018/D21S1435 | 3 | ΔF508/ΔF508 | ΔF508/ΔF508 | ΔF508/N | afflicted |
| 9^{a} | D16S539/D16S3018/D21S1435 | 3 | ΔF508/N | ΔF508/N | ΔF508/N | CF carrier |
| 10^{a} | D7S486/D21S1435/D21S1437 | 3 | ΔF508/N | ΔF508/N | ΔF508/N | CF carrier |
| 11^{de} | D7S486/D7S490/D7S523 | 4 | Mut/N | | Mut/N | CF carrier |
| 12^{d} | D7S480/D7S486/D7S523 | 3 | N/N | | N/N | healthy |

Abbreviations:
ISET: isolation by size of epithelial tumor/trophoblastic cells;
CVS: chorionic villus sampling;
ΔF508/N: with heterozygous ΔF508/N;
N/N: with normal homozygous alleles;
ΔF508/ΔF508: with homozygous ΔF508 alleles;
Mut/N: with mutated heterozygous allele.
^{a}ΔF508 carriers;
^{b}accurate amplification and coherent results obtained in all CFCs tested;
^{c}pooling of pre-amplification products by primer elongation (PEP) of three tested CFCs;
^{d}carriers of unknown mutations;
^{e}one parent is a ΔF508 carrier.

In circulating epithelial cells which have been shown to be fetal by STR genotyping, we investigated, on an individual basis and in groups of three cells, the presence of ΔF508 mutations. The test groups were produced, as described above, by mixing 30 μl of the PEP product of three fetal cells per blood sample and then by taking 12 μl (from 90 μl for F508 amplification). Sequence analyses were carried out using the Big Dye Terminator sequencing kit (Applied Biosystems) (Vona et al, 2002) after purification of the PCR product on MicroSpin S-400RH columns (Amersham Bioscience, Bucks, UK). In couples for whom neither or only one parent was a carrier of the ΔF508 mutation, fetal cell analysis was carried out by an indirect method using polymorphic informative STR markers linked to the CFTR locus on chromosome 7, allowing the index case and the fetus haplotypes to be compared.

Non-invasive tests were carried out in the Biochemistry department at the Hôpital Necker for sick children using a completely blind approach by operators who had not been informed of the results of the invasive analysis which was carried out at the Medical Genetics Department of the same hospital.

Statistical Analyses

The chi2 test was used for the statistical inter group frequency analysis. A p value of less than 0.05 was considered to be significant.

### 2-2 Results

Single Cell STR Genotyping

To optimize the STR genotyping protocol, we analyzed the genomes of 75 individual leukocytes using one or three heterozygous STR markers (M1, M2 and M3 (see Table 3 above). This allowed us to distinguish the two alleles and determine the allele drop-out (ADO) at the genotyping step (see Table 3). By using only one marker, M1 or M2 or M3, we obtained a PCR efficiency (number of cells producing a PCR product) of 77%, 72% and 69% respectively, and a PCR precision (number of cells with the two alleles) of 72%, 70% and 75% respectively. When we combined the results obtained with the three STR markers, the PCR efficiency (number of cells giving a PCR product with at least one of the three STR markers used) was 95% and the PCR precision (number of cells with two alleles with at least one of the three STR markers used) had increased significantly to 94% (see Table 3). These data show that in the step for genotyping individual micro-dissected cells, the efficiency and PCR precision were significantly enhanced by carrying out informative multi marker STR genotyping.

Amplification of F508 Locus of CFTR Gene

To develop a protocol which overcomes the problem of allele drop-out (ADO), we used the DNA from 75 individual leukocytes deriving from proven ΔF508 carriers. In this case, the normal allele and the mutated allele are distinguishable as they have different sizes (120 bp and 117 bp respectively) (see, for example, FIG. 5A′) in the individual cell amplifications; we detected at least one F508 allele in 67 of the 75 cells. In the other 8 cells, there was no signal, which indicated that the pre-amplification step was insufficient. Thus, the PCR efficiency was 90%. Of the 67 positive cells for PCR, we observed one allele drop-out (ADO) in six of these cells (9%) and a precise amplification of the two alleles in the 61 remaining cells, which produced a PCR precision of 91% (see Table 3).

Since allele drop-out is a stochastic event, and affects one or the other allele in a frequency of about 1 in 10 analyses (0.1), it will affect the same allele in a frequency of 1/20 (0.05). Thus, we deduced that if we were to mix the DNA of 3 or more fetal cells, the frequency of allele drop-out affecting the same allele would fall to 0.0001 (0.05×0.05×0.05=0.000125). Thus, we mixed, blind (by an operator not aware of the preceding results) the PEP products of 67 DNA samples from leukocytes in 30 randomly selected groups of three, and we carried out F508 amplification and analysis of the fragment as described above. In this case, we coherently observed the absence of allele drop-out (see Table 3 above) in all 30 tests.

When we compared the individual cell results with those of the mixed samples, we observed that in 3 cases we had mixed the DNA from two cells which, when analyzed individually, produced one allele drop-out and the DNA from one cell which when individually analyzed, had the two alleles. The result of mixing three samples corresponds to an absence of allele drop-out. In one case, we mixed three cells which individually resulted in one allele drop-out in the individual cell test but which when mixed together did not produce an allele drop-out. We then mixed the DNA of six cells, with one allele drop-out in nine randomly selected groups of three. The results show an absence of allele drop-out in all of the tests. We explain this observation by the fact that in the group analyses, the probabilities of including DNA sequences deriving from one (and the same) two alleles in the complete mixture is, as calculated above, very small ( 1/10000). We thus conclude that by mixing 30 μl of PEP products from three cells deriving from an individual fetus and taking 12 μl for PCR, we could markedly increase the probability of having bi-allele sequences and having a reliable test for detecting the presence of one ΔF508 allele in the non-invasive prenatal diagnosis of cystic fibrosis.

Protocol for Non-Invasive Prenatal Diagnosis of Cystic Fibrosis (FIG. 4)

Based on the results obtained, we defined the following protocol for the non-invasive prenatal diagnosis of cystic fibrosis. DNA extracted from maternal blood (1 ml) and paternal blood (1 ml) were compared using STR primers to identify informative STR markers. ISET was used to enrich the feto-circulating trophoblasts from maternal blood (5 ml). The trophoblastic cells, identified by KL1 immunomarking and by cell morphology, are then harvested individually from the ISET filter by laser capture micro-dissection (LCM) and their DNA is then analyzed after cell lysis. The complete genome of individual cells is firstly amplified by random primer elongation amplification (PEP) (final volume: 60 μl). Aliquots of the PEP product (4 μl) are used for genotyping with 3 informative STR markers to determine whether the DNA is of fetal origin (having both maternal and paternal alleles) or of maternal origin. The informative STR markers on chromosome 7 allow an indirect diagnosis of cystic fibrosis (if the index case is available). Aliquots (30 μl) of the PEP product from three fetal cells are mixed and 12 μl is used for amplification with specific F508 primers to avoid the risk of allele drop-out (ADO) and to reliably carry out the diagnosis of cystic fibrosis.

Application of Protocol to 12 Couples Risking having a Child with Cystic Fibrosis

We then applied this improved method to blood cells removed from mothers risking having a fetus afflicted with cystic fibrosis. The genotyping analyses allowed us to identify at least three fetal cells from 3 ml of each maternal blood (see FIG. 5, elements A, B and C), for example. These fetal cells were tested individually and also in groups. In groups 1 to 10 (see Table 4 above, see FIG. 5) where the two parents were carriers of ΔF508, we studied DNA extracted from fetal cells with primers covering the ΔF508 locus (see FIG. 5). This test allowed us to identify the ΔF508 mutation which is characterized by a deletion of three base pairs (CTT) which produces a PCR product of 117 bp instead of 120 bp (see FIGS. 5A′, B′ and C′). We then identified heterozygous fetuses carrying the ΔF508 mutation (couples 1, 3, 5, 7, 9, 10) (see FIG. 5, element A′), homozygous fetuses (couples 2, 4 and 6) (FIG. 5, element B′), and a mutant fetus homozygous for cystic fibrosis (couple 8) (FIG. 5, element C′). In six individual fetal cells, the results we obtained by genotyping were confirmed by sequencing the specific PCR product of the F508 locus (see FIG. 5, elements A″, B″, C″). Sequence analysis was not carried out systematically, given that the data obtained by genotyping were extremely clear. We obtained the same results by testing each CFC individually (see Table 4 above) and mixing the three CFCs identified in each maternal sample. The allele drop-out frequency was thus lower in samples deriving from CFCs (0/33) than that we observed for leukocytes (6/67). We did not observe allele drop-out in groups of PEP products starting from groups of PEP product from three fetal cells.

We also showed that we could carry out non-invasive prenatal diagnosis of cystic fibrosis in couples with unknown CFTR mutations provided that DNA from a sibling afflicted with cystic fibrosis (index case) was available to identify the STR alleles linked to the mutated CFTR alleles. In couple 11 (see Table 4), the father was a carrier of the ΔF508 mutation and the mother was a carrier of an unknown mutation (see FIG. 6, elements A, B and C). Analysis of individual CFCs with informative STR primers linked to the CFTR locus on chromosome 7 (indirect diagnosis) (see FIG. 6A) and by specific primers of the F508 locus (see FIG. 6B) showed an absence of the ΔF508 mutation and the heterozygous presence of the maternal allele carrying the unknown mutation which was identified by its presence in the genome of the index case. Hence, the fetus was a carrier of the unknown maternal mutation. In couple 12, the mother and father were carriers of the unknown mutation (see FIGS. 6D and E). Analyses of the fetal cells with informative STR primers on chromosome 7 (indirect diagnosis) showed the homozygous absence of the mutated alleles, previously identified in the genome of the index case. The fetus was thus completely normal.

The work was entirely carried out using a blind protocol and coherent results were obtained by the non invasive ISET-CF method and by the invasive method carried out by chorionic villus sampling (CVS) by an independent team (see Table 4 above).

### 3-3 Discussion

Our study shows that a non-invasive prenatal diagnosis of cystic fibrosis is possible and may be routinely applied in the context of clinical devices. In fact, clinical application of our method to fetal cells isolated from the blood of 12 mothers having a 25% risk of giving birth to a child afflicted with CFTR proved in a blind study to be a reliable diagnostic method in all cases with accurate identification of mothers with healthy or carrier or afflicted fetuses.

The method involves isolating circulating fetal cells (CFCs) by ISET and laser micro-dissection, genotyping to determine the presence of paternal markers (i.e. to confirm the fetal origin of these cells), and analysis of the mutation in a pool of three cells the fetal nature of which has been genetically proved. The fetal cells may be isolated from all mothers without a supplemental risk of miscarriage and the mutation may be analyzed using pools of cells the fetal nature of which has been proved, bringing to almost zero, and significantly, the probability of allele drop-out (ADO). These characteristics render the test reliable and potentially clinically applicable as a safe alternative to invasive prenatal diagnostic procedures.

Previous approaches to developing routine protocols for non-invasive prenatal diagnosis failed because of the low efficiency of the methods used to enrich and/or identify circulating fetal cells (CFCs) (Bianchi 1999; Bianchi et al, 2002). Further, tests on the fetus carrying a Y chromosome to identify fetal cells as being different from the maternal cell background were not applicable to female fetuses.

Fetal DNA free of cells in the maternal plasma corresponds to 3-5% of total plasma DNA (Lo et al, 1998) and allows the fetal sex, the rhesus D fetal condition and fetal point mutations inherited from the father to be determined as long as this DNA does not exist in the maternal genetic line (Li et al, 2005; Li et al, 2004). This approach, however, cannot be routinely applied for the prenatal diagnosis of recessive disorders such as cystic fibrosis which, by definition, requires a study of mutations inherited from the mother and the father.

Our novel approach overcomes all of the obstacles to the non-invasive prenatal diagnosis of cystic fibrosis. Rare circulating trophoblasts, which are considered not to persist after birth (Bianchi et al, 1996), are enriched by ISET as they are larger than peripheral blood leukocytes. This enrichment is more effective if the fetal cells, 3 or more, are found in a single sample of only 2-4 ml of blood from all mothers of a group of 51 tested so far in our laboratory. Individual cell genotyping after laser micro-dissection can identify fetal cells individually by means of the bi-parental contribution to their genome. On genotyping the individual cell, even if an ADO occurs, the consequence is that the DNA from the individual cell is incompletely characterized and as a result is removed. There is no risk of making an incorrect diagnosis. However, we show here that genotyping individual cells using 3 STR markers greatly reduces the risk of ADO by increasing the genotyping efficiency of the individual cell and accelerating the diagnostic process. Finally, the fact of pooling half of the primer elongation pre-amplification product (PEP) (30 μl of 60 μl) of 3 fetal cells and the fact of taking 12 μl for F508 amplification allows a mutation analysis to be carried out on “pure” fetal cell DNA, minimizing the false positive diagnosis due to the loss of a normal allele by ADO. It has been demonstrated that the mixture of DNA from 2 or more single cells almost completely removes the risk of ADO (Piyamongkol et al, 2003), but these studies were carried out on total cellular DNA (from fresh cells) which had not been amplified by PEP. In our protocol, we use aliquots of PEP products, rendering possible several PCR analyses on a genome from individual cells and on a pool of genomes from several cells starting from a single fetus.

In our experience, the optimized PEP protocol includes the degree of conservation of DNA (we store the ISET filters for non-invasive prenatal diagnosis at −20° C.), treatment with proteinase K to lyse the cell proteins (several protocols were comparatively tested) and the quality of the degenerate primers used for PEP (all of the batches of primers were verified before use in the non-invasive prenatal diagnosis protocol). We confirm that PEP does not impede the appearance of ADO (Hahn et al, 1998) but our results go further and show that ADO is impeded by mixing the mixed PEP products in the form of a large aliquot.

In fact, the results obtained by analyzing 75 test cells individually and in pools of 3 cells showed that the probability that one ADO, which occurs randomly, will fail amplification of the same allele in all of the three pooled cells is extremely small ( 1/10000). Provided that in routine protocols the non-invasive prenatal diagnosis could be repeated in 5 to 10 pools of 3 fetal cells from a 10 ml blood sample, and provided that the test could also be repeated in 2 supplemental blood samples taken from the same mother, the risk of an erroneous diagnosis could be close to zero.

In the single cell test, we observed a higher incidence of ADO in the 67 lymphocytes (9%) than in the 33 fetal cells (0%) analyzed by specific PCR for ΔF508. We believe that the compact nature of the DNA from lymphocytes could explain their higher ADO rate if the DNA from such cells is less accessible to PCR primers.

We show here that the non-invasive prenatal diagnosis of cystic fibrosis is possible even when both parents carry unknown CFTR mutations (indirect diagnosis) provided that DNA from an index case (an afflicted child from the same couple) is available and that informative STR primers on chromosome 7, where the CFTR gene is located, have been identified. In practice, DNA from an index case is generally available because the afflicted child is very often the only indication that the parents are carriers of CFTR mutations. In this indirect diagnosis, there is no need to pool fetal cells to avoid errors because the STR alleles required for the diagnosis, both maternal and paternal, have to be observed. Hence, the presence of two alleles ensures the absence of ADO and a reliable diagnosis.

In conclusion, we have developed a protocol based on ISET offering a reliable and safe prenatal diagnosis of the healthy condition, healthy carrier condition or condition of a carrier afflicted with cystic fibrosis.

This optimized strategy, which may be speeded up by automated laser micro-dissection and the development of kits for specific PCR analysis, should allow access to a non-invasive prenatal diagnosis of cystic fibrosis in clinical applications.

### 3-4 References for Example 4

Balinsky W, Zhu C W, 2004 Pediatric cystic fibrosis: evaluating costs and genetic testing. J Pediatr Health Care 18(1):30-4

Beroud C, Karliova M, Bonnefont J P, Benachi A, Munnich A, Dumez Y, Lacour B, Paterlini-Brechot P 2003. Prenatal diagnosis of spinal muscular atrophy by genetic analysis of circulating fetal cells. Lancet 361 (9362): 1013-4

Bianchi D W 1999, Fetal cells in the maternal circulation: feasibility for prenatal diagnosis. Br J Haematol 105(3):574-83

Bianchi D W, Simpson J L, Jackson L G, Elias S, Holzgreve W, Evans M I, Dukes K A, Sullivan L M, Klinger K W, Bischoff F Z and others, 2002. Fetal gender and aneuploidy detection using fetal cells in maternal blood: analysis of NIFTY I data National Institute of Child Health and Development Fetal Cell Isolation Study. Prenat Diagn 22(7):609-15

Bianchi D W, Zickwolf G K, Weil G J, Sylvester S, DeMaria M A. 1996 Male fetal progenitor cells persist in maternal blood for as long as 27 years postpartum Proc Natl Acad Sci USA 93(2):705-8

Ciarleglio L J, Bennett R L, Williamson J, Mandell J B, Marks J H, 2003. Genetic counseling throughout the life cycle. J Clin Invest 112(9): 1280-6

Fink L, Collins F S 1997. The Human Genome Project: view from the National Institutes of Health. J Am Med Womens Assoc 52(1):4-7, 15

Garvin A M, Holzgreve W, Hahn S, 1998. Highly accurate analysis of heterozygous loci by single cell PCR. Nucleic Acids Res 26(15):3468-72

Hahn S, Garvin A M, Di Naro E, Holzgreve W. 1998. Allele drop-out can occur in alleles differing by a single nucleotide and is not alleviated by preamplification or minor template increments. Genet Test 2(4):351-5

Hahn S, Zhong X Y, Holzgreve W, 2002. Single cell PCR in laser capture microscopy. Methods Enzymol 356:295-301

Hahn S, Zhong X Y, Troeger C, Burgemeister R, Gloning K, Holzgreve W, 2000. Current applications of single-cell PCR. Cell Mol Life Sci 57(1):96-105

Kerem B, Rommens J M, Buchanan J A, Markiewicz D, Cox T K, Chakravarti A, Buchwald M, Tsui L C, 1989. Identification of the cystic fibrosis gene: genetic analysis. Science 245(4922): 1073-80

Li Y, Di Naro E, Vitucci A, Zimmermann B, Holzgreve W, Hahn S 2005 Detection of paternally inherited fetal point mutations for beta-thalassemia using size-fractionated cell-free DNA in maternal plasma Jama 293(7):843-9

Li Y, Holzgreve W, Page-Christiaens G C, Gille J J, Hahn S, 2004. Improved prenatal detection of a fetal point mutation for achondroplasia by the use of size-fractionated circulatory DNA in maternal plasma-case report. Prenat Diagn 24(11):896-8

Lo Y M, Tein M S, Lau T K, Haines C J, Leung T N, Poon P M, Wainscoat J S, Johnson P J, Chang A M, Hjelm N M, 1998. Quantitative analysis of fetal DNA in maternal plasma and serum: implications for noninvasive prenatal diagnosis Am J Hum Genet 62(4):768-75

Nasis O, Thompson S, Hong T, Sherwood M, Radcliffe S, Jackson L, Otevrel T, 2004. Improvement in sensitivity of allele-specific PCR facilitates reliable noninvasive prenatal detection of cystic fibrosis. Clin Chem 50(4):694-701

Piyamongkol W, Bermudez M G, Harper J C, Wells D, 2003. Detailed investigation of factors influencing amplification efficiency and allele drop-out in single cell PCR: implications for preimplantation genetic diagnosis Mol Hum Reprod 9(7):411-20

Schmidtke J 1998 A Commentary on the NIH Consensus Development Statement ‘Genetic Testing for Cystic Fibrosis. Community Genet 1(1):53-6

Vona G, Beroud C, Benachi A, Quenette A, Bonnefont J P, Romana S, Dumez Y, Lacour B, Paterlini-Brechot P, 2002. Enrichment, immunomorphological, and genetic characterization of fetal cells circulating in maternal blood. Am J Pathol 160(1):51-8

Vona G, Sabile A, Louha M, Sitruk V, Romana S, Schutze K, Capron F, Franco D, Pazzagli M, Vekemans M and others, 2000. Isolation by size of epithelial tumor cells: a new method for the immunomorphological and molecular characterization of circulating tumor cells. Am J Pathol 156(1):57-63

Watson M S, Cutting G R, Desnick R J, Driscoll D A, Klinger K, Mennuti M, Palomaki G E, Popovich B W, Pratt V M, Rohlfs E M and others, 2004. Cystic fibrosis population carrier screening: 2004 revision of American College of Medical Genetics mutation panel Genet Med 6(5):387-91

Zhang L, Cui X, Schmitt K, Hubert R, Navidi W, Arnheim N, 1992 Whole genome amplification from a single cell: implications for genetic analysis Proc Natl Acad Sci USA 89(13):5847-51.

1. A non-invasive prenatal in vitro method for detecting the normal healthy condition, the healthy carrier condition or the condition of a carrier afflicted with cystic fibrosis, from a sample of fetal cell(s) isolated from a maternal sample which has been taken, comprising DNA to be tested from an individual, said method comprising the following steps:
a) enriching a pure or diluted maternal sample, which may include cells of fetal origin, in fetal cells;
b) analyzing the retained cells and selecting cells presumed to be of fetal origin;
c) demonstrating, by genetic analysis, the fetal origin of one or more cells selected in step b); and
d) on fetal DNA of cell(s) selected in step c), investigating alleles of the CFTR gene carrying the ΔF508 mutation or other known mutations, or investigating alleles of a locus carrying a genotypical polymorphism genetically linked to an unidentified morbid mutation of the CFTR gene by means of the following steps:
amplification of fetal DNA using pairs of primers selected for their capacity to amplify the locus which is capable of carrying the investigated known mutation on the CFTR gene or a locus comprising a genotypical polymorphism genetically linked (linkage) to the mutation of the CFTR gene during segregation;
identifying, on the alleles corresponding to the amplified DNA fragments, the presence or absence of the investigated known mutation of the CFTR gene or of the polymorphic locus genetically linked to the CFTR gene; and
comparing fetal alleles with alleles corresponding to control samples and determining, from observing the amplified alleles, detection of the normal healthy condition, the healthy carrier condition or the condition of a carrier afflicted with cystic fibrosis in a test individual.

2. A non-invasive prenatal in vitro method for detecting the normal healthy condition, the healthy carrier condition or the condition of a carrier afflicted with cystic fibrosis according to claim 1, in which the demonstration in step c) of the fetal origin of selected cells is carried out by genotyping said cells using the following steps:
i) amplifying individually taken DNA from one or more selected cells using primers termed informative primers, capable of amplifying predetermined genetic polymorphisms to distinguish maternal alleles from paternal alleles in order to recognize the fetal genome by the presence of a paternal allele and a maternal allele in each selected cell;
ii) comparing alleles of DNA from said cells with the corresponding parental alleles;
iii) selecting DNA from cell(s) comprising a maternal allele and a paternal allele for the identified genetic polymorphisms, demonstrating the fetal origin of the DNA from the cell(s).

3. A non-invasive prenatal in vitro method for detecting the normal healthy condition, the healthy carrier condition or the condition of a carrier afflicted with cystic fibrosis according to claim 1, in which the primers used in steps c) and d) are capable of amplifying a small quantity of DNA.

4. A non-invasive prenatal in vitro method for detecting the normal healthy condition, the healthy carrier condition or the condition of a carrier afflicted with cystic fibrosis according to claim 1, in which the primers used in steps c) and d) are capable of amplifying DNA from a single cell.

5. A non-invasive prenatal in vitro method for detecting the normal healthy condition, the healthy carrier condition or the condition of a carrier afflicted with cystic fibrosis according to claim 3, in which the primers used are capable of amplifying a quantity of DNA of less than 5 pg, in particular of the order of 2 pg.

6. A non-invasive prenatal in vitro method for detecting the normal healthy condition, the healthy carrier condition or the condition of a carrier afflicted with cystic fibrosis according to claim 1, in which the amplification step of step c) and/or the amplification step of step d) comprises a first amplification phase carried out with external primers and a second amplification phase carried out with internal primers.

7. A non-invasive prenatal in vitro method for detecting the normal healthy condition, the healthy carrier condition or the condition of a carrier afflicted with cystic fibrosis according to claim 1, in which the informative primers used in step c) are derived from the sequence of a chromosome selected from chromosome 16, chromosome 21 and chromosome 7.

8. A non-invasive prenatal in vitro method for detecting the normal healthy condition, the healthy carrier condition or the condition of a carrier afflicted with cystic fibrosis according to claim 7, in which the primers used are derived from the sequence of chromosome 7 and are close to the morbid allele for cystic fibrosis.

9. A non-invasive prenatal method for detecting the normal healthy condition, the healthy carrier condition or the condition of a carrier afflicted with cystic fibrosis according to claim 1, in which, in step d), at least one allele carrying the mutation of the ΔF508 locus of the CFTR gene is investigated and the test fetal DNA is amplified with primers capable of amplifying the ΔF508 locus of the CFTR gene.

10. A non-invasive prenatal in vitro method for detecting the normal healthy condition, the healthy carrier condition or the condition of a carrier afflicted with cystic fibrosis according to claim 1, in which primer pairs selected from the following are used to amplify the ΔF508 locus:
(F: 5′-TGGAGCCTTCAGAGGGTAAA-3′ SEQ ID NO: 25;
R: 5′-TGCATAATCAAAAAGTTTTCACA-3′ SEQ ID NO: 26);
and
(F: 5′-TCTGTTCTCAGTTTTCCTGG-3′ SEQ ID NO: 27;
R: 5′-TCTTACCTCTTCTAGTTGGC-3′ SEQ ID NO: 28).

11. A non-invasive prenatal in vitro method for detecting the normal healthy condition, the healthy carrier condition or the condition of a carrier afflicted with cystic fibrosis according to claim 1, in which the investigated anomaly in the CFTR gene is unknown and steps c) and d) are carried out under the following conditions:
the amplification of steps c) and d) is combined and carried out using informative amplification primers which are capable of amplifying a locus comprising a genotypical polymorphism genetically linked (linkage) to the CFTR gene during segregation; and
the comparison with the control samples of step d) comprises comparing the alleles identified from fetal DNA with the corresponding paternal alleles, the corresponding maternal alleles and the alleles corresponding to a child of the same parentage afflicted with cystic fibrosis.

12. A non-invasive prenatal in vitro method for detecting the normal healthy condition, the healthy carrier condition or the condition of a carrier afflicted with cystic fibrosis according to claim 1, to identify the presence or absence of at least one allele carrying a genetic polymorphism forming a linkage with the morbid allele for cystic fibrosis, at least one phase for amplifying fetal DNA, maternal DNA, paternal DNA and DNA from a child of the same parentage is carried out with one or more pairs of primers selected from:
(F: 5′-AAAAACCCTGGCTTATGG-3′ SEQ ID NO: 1;
R: 5′-AGCTACCATAGGGCTGGAGG-3′ SEQ ID NO: 2),
(F: 5′-GGAATCTGTTCTGGCAATGGAT-3′ SEQ ID NO: 5;
R: 5′-TTGCAATGAGCCGAGATCCTG-3′ SEQ ID NO: 6),
(F: 5′-AAGTAATTCTCCTGCCTCAG-3′ (SEQ ID NO: 29);
R:
5′-AGCTACTTGCAGTGTAACAGCATTT-3′ (SEQ ID NO: 30));
(F: 5′-GAATTATAACCGTAACTGATTC-3′ (SEQ ID NO: 33);
R: 5′-GAGATAATGCTTGTCTGACTTC-3′ (SEQ ID NO: 34));
(F: 5′-CTTGGGGACTGAACCATCTT-3′ SEQ ID NO: 3;
R: 5′-AGCTACCATAGGGCTGGAGG-3′ SEQ ID NO: 4),
(F: 5′-AAAGGCCAATGGTATATCCC-3′ SEQ ID NO: 7;
R: 5′-GCCCAGGTGATTGATAGTGC-3′ SEQ ID NO: 8),
(F: 5′-CCTTGGGGCCAATAAGGTAAG-3′ (SEQ ID NO: 31);
R:
5′-AGCTACTTGCAGTGTAACAGCATTT-3′ (SEQ ID NO: 32));
(F: 5′-CTGATTCATAGCAGCACTTG-3′ (SEQ ID NO: 35);
R: 5′-AAAACATTTCCATTACCACTG-3′ (SEQ ID NO: 36)).

13. A non-invasive prenatal in vitro method for detecting the normal healthy condition, the healthy carrier condition or the condition of a carrier afflicted with cystic fibrosis according to claim 1, in which step c) comprises or consists of identifying, on a DNA preparation derived from the genome of a single collected fetal cell, one or more genetic polymorphism markers or a combination of said markers, demonstrating the bi-parental contribution of the DNA from said cell and as a consequence, the fetal origin of said at least one cell.

14. A non-invasive prenatal in vitro method for detecting the normal healthy condition, the healthy carrier condition or the condition of a carrier afflicted with cystic fibrosis according to claim 13, in which the primer pairs termed informative primer pairs used in step c) are selected from:
SEQ ID NO: 1
F: 5′-AAAAACCCTGGCTTATGC-3′;
SEQ ID NO: 2
R: 5′-AGCTACCATAGGGCTGGAGG-3′,
SEQ ID NO: 5
F: 5′-GGAATCTGTTCTGGCAATGGAT-3′;
SEQ ID NO: 6
R: 5′-TTGCAATGAGCCGAGATCCTG-3′,
SEQ ID NO: 9
F: 5′-CAGATGCTCGTTGTGCACAA-3′;
SEQ ID NO: 10
R: 5′-ATACCATTTACGTTTGTGTGTG-3′,
SEQ ID NO: 13
F: 5′-TGACAGTGCAGCTCATGGTC-3′;
SEQ ID NO: 14
R: 5′-GGTCATTGGTCAAGGGCTGCT-3′;
SEQ ID NO: 17
F: 5′-TTGACATTCTTCTGTAAGGAAGA-3′;
SEQ ID NO: 18
R: 5′-AGGCTTGCCAAAGATATTAAAAG-3′,
SEQ ID NO: 21
F: 5′-TTGTGAATAGTGCTGCAATG-3′;
SEQ ID NO: 22
R: 5′-ATGTACACTGACTTGTTTGAG-3′,
(SEQ ID NO: 29)
F: 5′-AAGTAATTCTCCTGCCTCAG-3′;
(SEQ ID NO: 30)
R: 5′-AGCTACTTGCAGTGTAACAGCATTT-3′;
(SEQ ID NO: 33)
F: 5′-GAATTATAACCGTAACTGATTC-3′;
(SEQ ID NO: 34)
R: 5′-GAGATAATGCTTGTCTGACTTC-3′;
SEQ ID NO: 3
F: 5′-CTTGGGGACTGAACCATCTT-3′;
SEQ ID NO: 4
R: 5′-AGCTACCATAGGGCTGGAGG-3′,
SEQ ID NO: 7
F: 5′-AAAGGCCAATGGTATATCCC-3′;
SEQ ID NO: 8
R: 5′-GCCCAGGTGATTGATAGTGC-3′,
SEQ ID NO: 11
F: 5′-GATCCCAAGCTCTTCCTCTT-3′;
SEQ ID NO: 12
R: 5′-ACGTTTGTGTGTGCATCTGT-3′,
SEQ ID NO: 15
F: 5′-GGATAAACATAGAGCGACAGTTC-3′;
SEQ ID NO: 16
R: 5′-AGACAGAGTCCCAGGCATT-3′,
SEQ ID NO: 19
F: 5′-CCCTCTCAATTGTTTGTCTACC-3′;
SEQ ID NO: 20
R: 5′-GCAAGAGATTTCAGTGCCAT-3′,
SEQ ID NO: 23
F: 5′-ATGTACATGTGTCTGGGAAGG-3′;
SEQ ID NO: 24
R: 5′-TTCTCTACATATTTACTGCCAACA-3′,
(SEQ ID NO: 31)
F: 5′-CCTTGGGCCAATAAGGTAAG-3′;
(SEQ ID NO: 32)
R: 5′-AGCTACTTGCAGTGTAACAGCATTT-3′;
(SEQ ID NO: 35)
F: 5′-CTGATTCATAGCAGCACTTG-3′;
(SEQ ID NO: 36)
R: 5′-AAAACATTTCCATTACCACTG-3′.

15. A non-invasive prenatal in vitro method for detecting the normal healthy condition, the healthy carrier condition or the condition of a carrier afflicted with cystic fibrosis according to claim 11, in which the following primers are used in step c):
SEQ ID NO: 1
F: 5′-AAAAACCCTGGCTTATGC-3′,
SEQ ID NO: 2
R: 5′-AGCTACCATAGGGCTGGAGG-3′;
or
SEQ ID NO: 5
F: 5′-GGAATCTGTTCTGGCAATGGAT-3′,
SEQ ID NO: 6
R: 5′-TTGCAATGAGCCGAGATCCTG-3′;
or
(SEQ ID NO: 29)
F: 5′-AAGTAATTCTCCTGCCTCAG-3′,
(SEQ ID NO: 30)
R: 5′-AGCTACTTGCAGTGTAACAGCATTT-3′;
or
(SEQ ID NO: 33)
F: 5′-GAATTATAACCGTAACTGATTC-3′,
(SEQ ID NO: 34)
R: 5′-GAGATAATGCTTGTCTGACTTC-3′,
to carry out a first amplification phase;
SEQ ID NO: 3
F: 5′-CTTGGGGACTGAACCATCTT-3′,
SEQ ID NO: 4
R: 5′-AGCTACCATAGGGCTGGAGG-3′;
or
SEQ ID NO: 7
F: 5′-AAAGGCCAATGGTATATCCC-3′,
SEQ ID NO: 8
R: 5′-GCCCAGGTGATTGATAGTGC-3′;
or
(SEQ ID NO: 31)
F: 5′-CCTTGGGCCAATAAGGTAAG-3′,
(SEQ ID NO: 32)
R: 5′-AGCTACTTGCAGTGTAACAGCATTT-3′;
or
(SEQ ID NO: 35)
F: 5′-CTGATTCATAGCAGCACTTG-3′,
(SEQ ID NO: 36)
R: 5′-AAAACATTTCCATTACCACTG-3′
to carry out a second amplification phase.

16. A non-invasive prenatal in vitro method for detecting the normal healthy condition, the healthy carrier condition or the condition of a carrier afflicted with cystic fibrosis according to claim 10, in which the primers used which are capable of amplifying the ΔF508 locus are: F: 5′-TGGAGCCTTCAGAGGGTAAA-3′ (SEQ ID NO: 25), R: 5′-TGCATAATCAAAAAGTTTTCACA-3′ (SEQ ID NO: 26) for the first amplification phase and F: 5′-TCTGTTCTCAGTTTTCTGG-3′ (SEQ ID NO: 27), R: 5-TCTTACCTCTTCTAGTTGGC-3′ (SEQ ID NO: 28) for the second amplification phase.

17. A non-invasive prenatal in vitro method for detecting the normal healthy condition, the healthy carrier condition or the condition of a carrier afflicted with cystic fibrosis according to claim 1, in which the cells retained during step a) are collected individually, in particular by micro-dissection.

18. A non-invasive prenatal in vitro method for detecting the normal healthy condition, the healthy carrier condition or the condition of a carrier afflicted with cystic fibrosis according to claim 1, in which the cells retained during step a) are collected then analyzed in situ during step b) without collecting the cells individually.

19. A non-invasive prenatal in vitro method for detecting the normal healthy condition, the healthy carrier condition or the condition of a carrier afflicted with cystic fibrosis according to claim 1, in which step a) consists of filtering a pure or diluted maternal sample which may comprise cells of fetal origin, to concentrate on a filter according to size certain cells including cells of fetal origin, and step b) consists of analyzing cells retained on the filter and selecting cells presumed to be of fetal origin.

20. A non-invasive prenatal in vitro method for detecting the normal healthy condition, the healthy carrier condition or the condition of a carrier afflicted with cystic fibrosis according to claim 10, in which at least one primer which can be used to carry out step c) and/or step d) is replaced by a variant oligonucleotide the sequence of which is derived from that of one of said primers, said oligonucleotide variant having at least 60% identity, preferably 80% and more preferably 95% identity or more with the primer from which its sequence derives.

21. A non-invasive prenatal in vitro method for detecting the normal healthy condition, the healthy carrier condition or the condition of a carrier afflicted with cystic fibrosis according to claim 1, characterized in that said step d) is carried out on the pool of DNA from several cells selected individually in said step c).

22. A polynucleotide for use as a primer to amplify a quantity of DNA from a biological sample, characterized in that it is selected from polynucleotides the sequence of which comprises or consists of one of the following sequences:
SEQ ID NO: 1
F: 5′-AAAAACCCTGGCTTATGC-3′;
SEQ ID NO: 2
R: 5′-AGCTACCATAGGGCTGGAGG-3′;
SEQ ID NO: 5
F: 5′-GGAATCTGTTCTGGCAATGGAT-3′;
SEQ ID NO: 6
R: 5′-TTGCAATGAGCCGAGATCCTG-3′;
SEQ ID NO: 9
F: 5′-CAGATGCTCGTTGTGCACAA-3′;
SEQ ID NO: 10
R: 5′-ATACCATTTACGTTTGTGTGTG-3′;
SEQ ID NO: 13
F: 5′-TGACAGTGCAGCTCATGGTC-3′;
SEQ ID NO: 14
R: 5′-GGTCATTGGTCAAGGGCTGCT-3′;
SEQ ID NO: 17
F: 5′-TTGACATTCTTCTGTAAGGAAGA-3′;
SEQ ID NO: 18
R: 5′-AGGCTTGCCAAAGATATTAAAAG-3′;
SEQ ID NO: 21
F: 5′-TTGTGAATAGTGCTGCAATG-3′;
SEQ ID NO: 22
R: 5′-ATGTACACTGACTTGTTTGAG-3′;
(SEQ ID NO: 29)
F: 5′-AAGTAATTCTCCTGCCTCAG-3′;
(SEQ ID NO: 30)
R: 5′-AGCTACTTGCAGTGTAACAGCATTT-3′;
(SEQ ID NO: 33)
F: 5′-GAATTATAACCGTAACTGATTC-3′;
(SEQ ID NO: 34)
R: 5′-GAGATAATGCTTGTCTGACTTC-3′;
SEQ ID NO: 25
F: 5′-TGGAGCCTTCAGAGGGTAAA-3′;
SEQ ID NO: 26
R: 5′-TGCATAATCAAAAAGTTTTCACA-3′;
SEQ ID NO: 27
F: 5′-TCTGTTCTCAGTTTTCCTGG-3′;
SEQ ID NO: 28
R: 5′-TCTTACCTCTTCTAGTTGGC-3′.

23. A pair of polynucleotides for use as a pair of primers to amplify a quantity of DNA from a biological sample, characterized in that it is selected from pairs of polynucleotides the sequences of which comprise or consist of:
SEQ ID NO: 1
F: 5′-AAAAACCCTGGCTTATGC-3′;
SEQ ID NO: 2
R: 5′-AGCTACCATAGGGCTGGAGG-3′;
SEQ ID NO: 5
F: 5′-GGAATCTGTTCTGGCAATGGAT-3′;
SEQ ID NO: 6
R: 5′-TTGCAATGAGCCGAGATCCTG-3′;
SEQ ID NO: 9
F: 5′-CAGATGCTCGTTGTGCACAA-3′;
SEQ ID NO: 10
R: 5′-ATACCATTTACGTTTGTGTGTG-3′;
SEQ ID NO: 13
F: 5′-TGACAGTGCAGCTCATGGTC-3′,
SEQ ID NO: 14
R: 5′-GGTCATTGGTCAAGGGCTGCT-3′;
SEQ ID NO: 17
F: 5′-TTGACATTCTTCTGTAAGGAAGA-3′;
SEQ ID NO: 18
R: 5′-AGGCTTGCCAAAGATATTAAAAG-3′;
SEQ ID NO: 21
F: 5′-TTGTGAATAGTGCTGCAATG-3′;
SEQ ID NO: 22
R: 5′-ATGTACACTGACTTGTTTGAG-3′;
(SEQ ID NO: 29)
F: 5′-AAGTAATTCTCCTGCCTCAG-3′;
(SEQ ID NO: 30)
R: 5′-AGCTACTTGCAGTGTAACAGCATTT-3′;
(SEQ ID NO: 33)
F: 5′-GAATTATAACCGTAACTGATTC-3′;
(SEQ ID NO: 34)
R: 5′-GAGATAATGCTTGTCTGACTTC-3′;
SEQ ID NO: 25
F: 5′-TGGAGCCTTCAGAGGGTAAA-3′;
SEQ ID NO: 26
R: 5′-TGCATAATCAAAAAGTTTTCACA-3′;
SEQ ID NO: 27
F: 5′-TCTGTTCTCAGTTTTCCTGG-3′;
SEQ ID NO: 28
R: 5′-TCTTACCTCTTCTAGTTGGC-3′.

24. A polynucleotide having at least 60% identity, preferably 80%, more preferably 95% identity or more with a sequence according to claim 22.

25. An association of a pair of primers, termed external primers with respect to the target sequence, used in a first phase for amplification of a DNA preparation, and another pair of primers, termed internal or nested primers with respect to said external primers, used in a second amplification phase on the amplification product obtained by said first amplification phase, said pairs being selected from:

| | |
|---|---|
| F: 5′-AAAAACCCTGGCTTATGC-3′; | SEQ ID NO: 1 |
| R: 5′-AGCTACCATAGGGCTGGAGG-3′ | SEQ ID NO: 2) |

as external primers; and

| | |
|---|---|
| F: 5′-CTTGGGGACTGAACCATCTT-3′; | SEQ ID NO: 3 |
| R: 5′-AGCTACCATAGGGCTGGAGG-3′ | SEQ ID NO: 4 |

as internal primers; or

| | |
|---|---|
| F: 5′-GGAATCTGTTCTGGCAATGGAT-3′; | SEQ ID NO: 5 |
| R: 5′-TTGCAATGAGCCGAGATCCTG-3′ | SEQ ID NO: 6 |

as external primers and

| | |
|---|---|
| F: 5′-AAAGGCCAATGGTATATCCC-3′; | SEQ ID NO: 7 |
| R: 5′-GCCCAGGTGATTGATAGTGC-3′ | SEQ ID NO: 8 |

as internal primers; or

| | |
|---|---|
| F: 5′-CAGATGCTCGTTGTGCACAA-3′; | SEQ ID NO: 9 |
| R: 5′-ATACCATTTACGTTTGTGTGTG-3′ | SEQ ID NO: 10 |

as external primers; and

| | |
|---|---|
| F: 5′-GATCCCAAGCTCTTCCTCTT-3′; | SEQ ID NO: 11 |
| R: 5′-ACGTTTGTGTGTGCATCTGT-3′ | SEQ ID NO: 12 |

as internal primers; or

| | |
|---|---|
| F: 5′-TGACAGTGCAGCTCATGGTC-3′, | SEQ ID NO: 13 |
| R: 5′-GGTCATTGGTCAAGGGCTGCT-3′; | SEQ ID NO: 14 |

as external primers; and

| | |
|---|---|
| F: 5′-GGATAAACATAGAGCGACAGTTC-3′; | SEQ ID NO: 15 |
| R: 5′-AGACAGAGTCCCAGGCATT-3′ | SEQ ID NO: 16 |

as internal primers; or

| | |
|---|---|
| F: 5′-TTGACATTCTTCTGTAAGGAAGA-3′; | SEQ ID NO: 17 |
| R: 5′-AGGCTTGCCAAAGATATTAAAAG-3′ | SEQ ID NO: 18 |

as external primers; and

| | |
|---|---|
| F: 5′-CCCTCTCAATTGTTTGTCTACC-3′; | SEQ ID NO: 19 |
| R: 5′-GCAAGAGATTTCAGTGCCAT-3′ | SEQ ID NO: 20 |

as internal primers; or

| | |
|---|---|
| F: 5′-TTGTGAATAGTGCTGCAATG-3′; | SEQ ID NO: 21 |
| R: 5′-ATGTACACTGACTTGTTTGAG-3′ | SEQ ID NO: 22 |

as external primers; and

| | |
|---|---|
| F: 5′-ATGTACATGTGTCTGGGAAGG-3′; | SEQ ID NO: 23 |
| R: 5′-TTCTCTACATATTTACTGCCAACA-3′ | SEQ ID NO: 24 |

as internal primers; or

| | |
|---|---|
| F: 5′-TGGAGCCTTCAGAGGGTAAA-3′; | SEQ ID NO: 25 |
| R: 5′-TGCATAATCAAAAAGTTTTCACA-3′ | SEQ ID NO: 26 |

as external primers; and

| | |
|---|---|
| F: 5′-TCTGTTCTCAGTTTTCCTGG-3′; | SEQ ID NO: 27 |
| R: 5′-TCTTACCTCTTCTAGTTGGC-3′ | SEQ ID NO: 28 |

as internal primers; or

| | |
|---|---|
| F: 5′-AAGTAATTCTCCTGCCTCAG-3′; | (SEQ ID NO: 29) |
| R: 5′-AGCTACTTGCAGTGTAACAGCATTT-3′ | (SEQ ID NO: 30) |

as external primers; and

| | |
|---|---|
| F: 5′-CCTTGGGCCAATAAGGTAAG-3′; | (SEQ ID NO: 31) |
| R: 5′-AGCTACTTGCAGTGTAACAGCATTT-3′ | (SEQ ID NO: 32) |

as internal primers; or

| | |
|---|---|
| F: 5′-GAATTATAACCGTAACTGATTC-3′; | (SEQ ID NO: 33) |
| R: 5′-GAGATAATGCTTGTCTGACTTC-3′ | (SEQ ID NO: 34) |

as external primers; and

| | |
|---|---|
| F: 5′-CTGATTCATAGCAGCACTTG-3′; | (SEQ ID NO: 35) |
| R: 5′-AAAACATTTCCATTACCACTG-3′ | (SEQ ID NO: 36) |

as internal primers.

26. Use of primers in the context of a non-invasive prenatal in vitro method for detecting the normal healthy condition, the healthy carrier condition or the condition of a carrier afflicted with cystic fibrosis from genomic DNA of fetal cells isolated from a maternal sample, characterized in that the primers are selected from:
SEQ ID NO: 1
F: 5′-AAAAACCCTGGCTTATGC-3′;
SEQ ID NO: 2
R: 5′-AGCTACCATAGGGCTGGAGG-3′;
SEQ ID NO: 5
F: 5′-GGAATCTGTTCTGGCAATGGAT-3′;
SEQ ID NO: 6
R: 5′-TTGCAATGAGCCGAGATCCTG-3′;
(SEQ ID NO: 29)
F: 5′-AAGTAATTCTCCTGCCTCAG-3′;
(SEQ ID NO: 30)
R: 5′-AGCTACTTGCAGTGTAACAGCATTT-3′;
(SEQ ID NO: 33)
F: 5′-GAATTATAACCGTAACTGATTC-3′;
(SEQ ID NO: 34)
R: 5′-GAGATAATGCTTGTCTGACTTC-3′;
SEQ ID NO: 3
F: 5′-CTTGGGGACTGAACCATCTT-3′;
SEQ ID NO: 4
R: 5′-AGCTACCATAGGGCTGGAGG-3′;
SEQ ID NO: 7
F: 5′-AAAGGCCAATGGTATATCCC-3′;
SEQ ID NO: 8
R: 5′-GCCCAGGTGATTGATAGTGC-3′;
(SEQ ID NO: 31)
F: 5′-CCTTGGGCCAATAAGGTAAG-3′;
(SEQ ID NO: 32)
R: 5′-AGCTACTTGCAGTGTAACAGCATTT-3′;
(SEQ ID NO: 35)
F: 5′-CTGATTCATAGCAGCACTTG-3′;
(SEQ ID NO: 36)
R: 5′-AAAACATTTCCATTACCACTG-3′;
SEQ ID NO: 25
F: 5′-TGGAGCCTTCAGAGGGTAAA-3′;
SEQ ID NO: 26
R: 5′-TGCATAATCAAAAAGTTTTCACA-3′;
SEQ ID NO: 27
F: 5′-TCTGTTCTCAGTTTTCCTGG-3′;
SEQ ID NO: 28
R: (5′-TCTTACCTCTTCTAGTTGGC-3′.

27. Use of primers in the context of an in vitro method for identifying the fetal nature of a single cell collected from a maternal sample on a preparation of DNA derived from the genome of the single collected cell, characterized in that the primers are selected from:
SEQ ID NO: 1
F: 5′-AAAAACCCTGGCTTATGC-3′;
SEQ ID NO: 2
R: 5′-AGCTACCATAGGGCTGGAGG-3′;
SEQ ID NO: 5
F: 5′-GGAATCTGTTCTGGCAATGGAT-3′;
SEQ ID NO: 6
R: 5′-TTGCAATGAGCCGAGATCCTG-3′;
SEQ ID NO: 9
F: 5′-CAGATGCTCGTTGTGCACAA-3′;
SEQ ID NO: 10
R: 5′-ATACCATTTACGTTTGTGTGTG-3′;
SEQ ID NO: 13
F: 5′-TGACAGTGCAGCTCATGGTC-3′,
SEQ ID NO: 14
R: 5′-GGTCATTGGTCAAGGGCTGCT-3′;
SEQ ID NO: 17
F: 5′-TTGACATTCTTCTGTAAGGAAGA-3′;
SEQ ID NO: 18
R: 5′-AGGCTTGCCAAAGATATTAAAAG-3′;
SEQ ID NO: 21
F: 5′-TTGTGAATAGTGCTGCAATG-3′;
SEQ ID NO: 22
R: 5′-ATGTACACTGACTTGTTTGAG-3′;
(SEQ ID NO: 29)
F: 5′-AAGTAATTCTCCTGCCTCAG-3′;
(SEQ ID NO: 30)
R: 5′-AGCTACTTGCAGTGTAACAGCATTT-3′;
(SEQ ID NO: 33)
F: 5′-GAATTATAACCGTAACTGATTC-3′;
(SEQ ID NO: 34)
R: 5′-GAGATAATGCTTGTCTGACTTC-3′;
SEQ ID NO: 3
F: 5′-CTTGGGGACTGAACCATCTT-3′;
SEQ ID NO: 4
R: 5′-AGCTACCATAGGGCTGGAGG-3′;
SEQ ID NO: 7
F: 5′-AAAGGCCAATGGTATATCCC-3′;
SEQ ID NO: 8
R: 5′-GCCCAGGTGATTGATAGTGC-3′;
SEQ ID NO: 11
F: 5′-GATCCCAAGCTCTTCCTCTT-3′;
SEQ ID NO: 12
R: 5′-ACGTTTGTGTGTGCATCTGT-3′;
SEQ ID NO: 15
F: 5′-GGATAAACATAGAGCGACAGTTC-3′;
SEQ ID NO: 16
R: 5′-AGACAGAGTCCCAGGCATT-3′;
SEQ ID NO: 19
F: 5′-CCCTCTCAATTGTTTGTCTACC-3′;
SEQ ID NO: 20
R: 5′-GCAAGAGATTTCAGTGCCAT-3′;
SEQ ID NO: 23
F: 5′-ATGTACATGTGTCTGGGAAGG-3′;
SEQ ID NO: 24
R: 5′-TTCTCTACATATTTACTGCCAACA-3′;
(SEQ ID NO: 31)
F: 5′-CCTTGGGCCAATAAGGTAAG-3′;
(SEQ ID NO: 32)
R: 5′-AGCTACTTGCAGTGTAACAGCATTT-3′;
(SEQ ID NO: 35)
F: 5′-CTGATTCATAGCAGCACTTG-3′;
(SEQ ID NO: 36)
R: 5′-AAAACATTTCCATTACCACTG-3′.

28. A kit for the non-invasive prenatal detection of the normal healthy condition, the healthy carrier condition or the condition of a carrier afflicted with cystic fibrosis, comprising one or more primers in accordance with claim 27 and, if appropriate, reagents for amplifying DNA and/or instructions for detecting the healthy carrier condition or the condition of a carrier afflicted with cystic fibrosis.


SEQUENCE LISTING (36 sequences)

SEQ ID NO: 1 - DNA, 18 bp, Artificial Sequence
  Description of Artificial Sequence Synthetic primer
aaaaaccctggcttatgc

SEQ ID NO: 2 - DNA, 20 bp, Artificial Sequence
  Description of Artificial Sequence Synthetic primer
agctaccatagggctggagg

SEQ ID NO: 3 - DNA, 20 bp, Artificial Sequence
  Description of Artificial Sequence Synthetic primer
cttggggactgaaccatctt

SEQ ID NO: 4 - DNA, 20 bp, Artificial Sequence
  Description of Artificial Sequence Synthetic primer
agctaccatagggctggagg

SEQ ID NO: 5 - DNA, 22 bp, Artificial Sequence
  Description of Artificial Sequence Synthetic primer
ggaatctgttctggcaatggat

SEQ ID NO: 6 - DNA, 21 bp, Artificial Sequence
  Description of Artificial Sequence Synthetic primer
ttgcaatgagccgagatcctg

SEQ ID NO: 7 - DNA, 20 bp, Artificial Sequence
  Description of Artificial Sequence Synthetic primer
aaaggccaatggtatatccc

SEQ ID NO: 8 - DNA, 20 bp, Artificial Sequence
  Description of Artificial Sequence Synthetic primer
gcccaggtgattgatagtgc

SEQ ID NO: 9 - DNA, 20 bp, Artificial Sequence
  Description of Artificial Sequence Synthetic primer
cagatgctcgttgtgcacaa

SEQ ID NO: 10 - DNA, 22 bp, Artificial Sequence
  Description of Artificial Sequence Synthetic primer
ataccatttacgtttgtgtgtg

SEQ ID NO: 11 - DNA, 20 bp, Artificial Sequence
  Description of Artificial Sequence Synthetic primer
gatcccaagctcttcctctt

SEQ ID NO: 12 - DNA, 20 bp, Artificial Sequence
  Description of Artificial Sequence Synthetic primer
acgtttgtgtgtgcatctgt

SEQ ID NO: 13 - DNA, 20 bp, Artificial Sequence
  Description of Artificial Sequence Synthetic primer
tgacagtgcagctcatggtc

SEQ ID NO: 14 - DNA, 21 bp, Artificial Sequence
  Description of Artificial Sequence Synthetic primer
ggtcattggtcaagggctgct

SEQ ID NO: 15 - DNA, 23 bp, Artificial Sequence
  Description of Artificial Sequence Synthetic primer
ggataaacatagagcgacagttc

SEQ ID NO: 16 - DNA, 19 bp, Artificial Sequence
  Description of Artificial Sequence Synthetic primer
agacagagtcccaggcatt

SEQ ID NO: 17 - DNA, 23 bp, Artificial Sequence
  Description of Artificial Sequence Synthetic primer
ttgacattcttctgtaaggaaga

SEQ ID NO: 18 - DNA, 23 bp, Artificial Sequence
  Description of Artificial Sequence Synthetic primer
aggcttgccaaagatattaaaag

SEQ ID NO: 19 - DNA, 22 bp, Artificial Sequence
  Description of Artificial Sequence Synthetic primer
ccctctcaattgtttgtctacc

SEQ ID NO: 20 - DNA, 20 bp, Artificial Sequence
  Description of Artificial Sequence Synthetic primer
gcaagagatttcagtgccat

SEQ ID NO: 21 - DNA, 20 bp, Artificial Sequence
  Description of Artificial Sequence Synthetic primer
ttgtgaatagtgctgcaatg

SEQ ID NO: 22 - DNA, 21 bp, Artificial Sequence
  Description of Artificial Sequence Synthetic primer
atgtacactgacttgtttgag

SEQ ID NO: 23 - DNA, 21 bp, Artificial Sequence
  Description of Artificial Sequence Synthetic primer
atgtacatgtgtctgggaagg

SEQ ID NO: 24 - DNA, 24 bp, Artificial Sequence
  Description of Artificial Sequence Synthetic primer
ttctctacatatttactgccaaca

SEQ ID NO: 25 - DNA, 20 bp, Artificial Sequence
  Description of Artificial Sequence Synthetic primer
tggagccttcagagggtaaa

SEQ ID NO: 26 - DNA, 23 bp, Artificial Sequence
  Description of Artificial Sequence Synthetic primer
tgcataatcaaaaagttttcaca

SEQ ID NO: 27 - DNA, 20 bp, Artificial Sequence
  Description of Artificial Sequence Synthetic primer
tctgttctcagttttcctgg

SEQ ID NO: 28 - DNA, 20 bp, Artificial Sequence
  Description of Artificial Sequence Synthetic primer
tcttacctcttctagttggc

SEQ ID NO: 29 - DNA, 20 bp, Artificial Sequence
  Description of Artificial Sequence Synthetic primer
aagtaattctcctgcctcag

SEQ ID NO: 30 - DNA, 25 bp, Artificial Sequence
  Description of Artificial Sequence Synthetic primer
agctacttgcagtgtaacagcattt

SEQ ID NO: 31 - DNA, 20 bp, Artificial Sequence
  Description of Artificial Sequence Synthetic primer
ccttgggccaataaggtaag

SEQ ID NO: 32 - DNA, 25 bp, Artificial Sequence
  Description of Artificial Sequence Synthetic primer
agctacttgcagtgtaacagcattt

SEQ ID NO: 33 - DNA, 22 bp, Artificial Sequence
  Description of Artificial Sequence Synthetic primer
gaattataaccgtaactgattc

SEQ ID NO: 34 - DNA, 22 bp, Artificial Sequence
  Description of Artificial Sequence Synthetic primer
gagataatgcttgtctgacttc

SEQ ID NO: 35 - DNA, 20 bp, Artificial Sequence
  Description of Artificial Sequence Synthetic primer
ctgattcatagcagcacttg

SEQ ID NO: 36 - DNA, 21 bp, Artificial Sequence
  Description of Artificial Sequence Synthetic primer
aaaacatttccattaccactg